(19) United States
(12) Patent Application Publication
(10) Pub. No.: US 2014/0343358 A1
(43) Pub. Date: Nov. 20, 2014

(54) SECONDARY IMAGING ENDOSCOPIC DEVICE

(71) Applicant: Avantis Medical Systems, Inc., Sunnyvale, CA (US)

(72) Inventors: Salmaan HAMEED, San Jose, CA (US); Max Shuichi DANGERFIELD, Sunnyvale, CA (US)

(73) Assignee: Avantis Medical Systems, Inc., Sunnyvale, CA (US)

(21) Appl. No.: 14/280,469

(22) Filed: May 16, 2014

Related U.S. Application Data
(60) Provisional application No. 61/988,074, filed on May 2, 2014.
(60) Provisional application No. 61/902,079, filed on Nov. 8, 2013.
(60) Provisional application No. 61/824,933, filed on May 17, 2013.

(51) Int. Cl. A61B 1/05; A61B 1/00; A61B 1/012

(52) U.S. Cl. CPC A61B 1/053; A61B 1/012; A61B 1/0014; A61B 1/00179
USPC 600/109; 600/158

(57) ABSTRACT

Described herein are various detachable secondary imaging endoscopic devices that can be used in conjunction with an endoscope to provide additional fields of view so that multiple regions of a body cavity may be imaged simultaneously. In one variation, a secondary imaging endoscopic device comprises an endoscope attachment member configured to be disposed over an endoscope, a first imaging element and a corresponding first light source at a first location on the endoscope attachment member, and a second imaging element and a second light source at a second location that is adjacent to the first location. In some variations, a secondary imaging device comprises a fluid delivery module having one or more ports for fluid delivery. The multiple simultaneous images acquired by the secondary imaging endoscopic device imaging elements and the main endoscope imaging element can be combined or arranged together to form a continuous view of a body cavity.

### CROSS-REFERENCE TO RELATED APPLICATIONS

This application claims priority to U.S. Provisional Patent Application No. 61/988,074, filed May 2, 2014, U.S. Provisional Patent Application No. 61/902,079, filed Nov. 8, 2013, and U.S. Provisional Patent Application No. 61/824,933, filed May 17, 2013, each of which is hereby incorporated by reference in its entirety.

### BACKGROUND

Endoscopes are used in diagnostic and/or therapeutic procedures to access and image internal body cavities. Images acquired by endoscopes may be used to identify abnormalities in otherwise inaccessible regions of the body, and may also provide a conduit through which therapeutic agents or procedures may be applied to that region.

For example, a colonoscope is an endoscope that is used to examine the internal surfaces of the lower gastrointestinal tract. Images acquired by the colonoscope may be used to identify a polyp in the intestine. Once a polyp is visualized by the colonoscope, a surgical tool may be inserted through the working lumen of the colonoscope in order to biopsy the polyp for testing and/or remove the polyp, if so desired.

### BRIEF SUMMARY

Internal body cavities often have irregular geometries and surface properties that may interfere with imaging and accessing tissues of interest. For example, since the gastrointestinal tract is tortuous and has a convoluted surface that includes many folds and pockets, it may be difficult for a practitioner to identify polyps and to contact a detected polyp with a surgical tool. Accordingly, improved endoscopy devices that provide additional views and/or facilitate the insertion of surgical tools may be desirable for diagnostic and/or therapeutic purposes.

Disclosed herein are secondary imaging devices that may be used in conjunction with an endoscope to provide additional fields of view so that multiple regions of a body cavity may be imaged simultaneously. A secondary imaging device may be attached to a distal portion of an endoscope, and may comprise one or more imaging elements that each have a different field of view. In some variations, the views acquired by the one or more imaging elements may overlap with each other and/or with the view acquired by the main endoscope imaging element, while in other variations, the views may not overlap with each other and/or with the view acquired by the main endoscope. The images acquired by the secondary imaging endoscopic device and the endoscope may be arranged and/or combined such that a practitioner is able to obtain a continuous view of a region of the body cavity. The acquired images may be displayed on one more displays, and/or may be digitally combined (e.g., stitched together) to create a continuous view of the body cavity. In some variations, a surgical tool (such as snare, cutter, and the like) may be inserted through a working lumen of the endoscope or the secondary imaging endoscopic device to contact and/or manipulate a tissue of interest. The secondary imaging endoscopic device may be disposed after each procedure, or reused for additional procedures. In some variations, the secondary imaging endoscopic device may be disposed after a certain number of procedures have been performed. Although the embodiments described herein may illustrate a secondary imaging endoscopic device for use with a colonoscope, it should be understood that such a device may be used with other types of endoscopes, including but not limited to a sigmoidoscope, a gastrointestinal endoscope, or endoscope used with endoscopic retrograde cholangiopancreatography (ERCP), as well as non-GI endoscopes, e.g. a ureteroscope, a cystoscope, and a uterine endoscope.

One variation of an imaging device for use with an endoscope having a front-facing imaging element may comprise a sleeve configured to be releasably disposed over a distal portion of the endoscope, a first side imaging element at a first circumferential location on the outer surface of the sleeve and having a visual axis that is tangential to the circumference of the sleeve, a first side light source located adjacent to the first imaging element such that the first light source provides illumination for the acquisition of images by the first imaging element, a second side imaging element at a second circumferential location on the outer surface of the sleeve and having a visual axis that is tangential to the circumference of the sleeve and co-linear with the visual axis of the first side imaging element, a second side light source located on the outer surface of the sleeve and adjacent to the second imaging element such that the second light source provides illumination for the acquisition of images by the second imaging element, and a fluid delivery module releasably mounted to the sleeve. The fluid delivery module may comprise a first outlet port and a second outlet port, where the first port is located adjacent to the first side imaging element and the second port is located adjacent to the second side imaging element. The first and second side imaging elements and the endoscope imaging element may be configured to simultaneously acquire images with different fields of view, and the fields of view of the images from the first and second side imaging elements may overlap with the field of view of the endoscope imaging element. In some variations, the first and second circumferential locations are adjacent to each other. Optionally, the sleeve may comprise first and second concave recesses and the first and second side imaging elements may each be located in the first and second concave recesses respectively. The curvature of each of the first and second concave recesses may permit at least a 135 degree viewing angle for each of the first and second side imaging elements, and optionally, the first and second light sources may each located in third and fourth recesses in the sleeve. In some variations, the first side imaging element and the second side imaging element may face in opposite directions. The fluid delivery module may comprise a housing, where one or more portions of the housing is optically translucent. The fluid delivery module may comprise a first inlet port and a conduit within the housing, and the conduit may connect the first inlet port with the first and second outlet ports. The housing of the fluid delivery module may comprise one or more curves around the first and second outlet ports that may form a fluid dynamic path toward the first and second side imaging elements for fluids exiting the first and second outlet ports. Optionally, the sleeve may comprise first and second concave recesses adjacent to the first and second side imaging devices such that the recesses are continuous with the fluid dynamic path formed by the one or more curves around the first and second outlet ports of the fluid delivery module.

The imaging device may further comprise a controller that is configured to combine the images acquired by the first and second side imaging element and the endoscope imaging element to simulate a continuous view. For example, the controller may be configured to stitch the images acquired by the first and second side imaging element and the endoscope imaging element into a single image having a continuous view. Optionally, the controller may be configured to output the images acquired by the first and second side imaging element and the endoscope imaging element to one or more display devices.

Another variation of an imaging device for use with an endoscope having a front-facing imaging element may comprise a sleeve configured to be releasably disposed over a distal portion of the endoscope, a first side imaging element at a first circumferential location on the outer surface of the sleeve and having a visual axis that is tangential to the circumference of the sleeve, a first side light source located directly adjacent to the first imaging element such that the first light source provides illumination for the acquisition of images by the first imaging element, a second side imaging element at a second circumferential location on the outer surface of the sleeve and having a visual axis that is tangential to the circumference of the sleeve and co-linear with the visual axis of the first side imaging element, a second side light source located on the outer surface of the sleeve and directly adjacent to the second imaging element such that the second light source provides illumination for the acquisition of images by the second imaging element, and a fluid delivery module releasably mounted to the sleeve. The fluid delivery module may comprise a first outlet port and a second outlet port, where the first port is located proximal to the first side light source and the second port is located proximal to the second light source. The first and second side imaging elements and the endoscope imaging element may be configured to simultaneously acquire images with different fields of view, and the fields of view of the images from the first and second side imaging elements may overlap with the field of view of the endoscope imaging element. In some variations, the first and second circumferential locations are adjacent to each other. In some variations, the first side imaging element and the second side imaging element may face in opposite directions. The fluid delivery module may comprise a housing, where one or more portions of the housing is optically translucent. The fluid delivery module may comprise a first inlet port and a conduit within the housing, and the conduit may connect the first inlet port with the first and second outlet ports. The housing of the fluid delivery module may comprise one or more curves around the first and second outlet ports that may form a fluid dynamic path toward the first and second side imaging elements for fluids exiting the first and second outlet ports.

Another variation of an imaging device for use with an endoscope having a front-facing imaging element may comprise a sleeve configured to be releasably disposed over a distal portion of the endoscope, a top-facing imaging element at a first circumferential location on the outer surface of the sleeve and having a visual axis that is perpendicular to the circumference of the sleeve and the visual axis of the front-facing imaging element, and a top-facing light source located directly adjacent to the first imaging element such that the light source provides illumination for the acquisition of images by the imaging element. The top-facing imaging element and the endoscope front-facing imaging elements may be configured to simultaneously acquire images with different fields of view, and the field of view of the images from the top-facing imaging element may overlap with the field of view of the endoscope front-facing imaging element.

Another variation of a secondary endoscopic imaging device (i.e., a detachable imaging device for use with an endoscope having a front-facing imaging element) may comprise a clip configured to be releasably disposed over a distal portion of an endoscope, the clip comprising a proximal edge, a distal edge, an inner region and an outer region, an imaging module attached to the outer region of the clip, a control cable attached to the imaging module for powering and controlling the imaging module separately from the endoscope, a fluid delivery module attached to the clip comprising a first outlet port, a second outlet port and an internal channel connecting the first and second outlet ports, and a fluid conduit connected to the internal channel of the fluid delivery module. The imaging module may comprise a first side-facing imaging element having a first visual axis, a first side light source adjacent to the first imaging element such that the first light source provides illumination for the acquisition of images by the first imaging element, a second side-facing imaging element having a second visual axis that is co-linear with the visual axis of the first side imaging element, and a second side light source located adjacent to the second imaging element such that the second light source provides illumination for the acquisition of images by the second imaging element. The first port of the fluid delivery module may be located proximal to the first side-facing imaging element and the second port may be located proximal to the second side-facing imaging element. The first and second outlet ports of the fluid delivery module may be each located within first and second concave regions of the fluid delivery module. The concavity of the first and second concave regions may be selected such that fluid from the first and second outlet ports are directed towards the first and second side-facing imaging elements. The fluid delivery module may further comprise an inlet port in communication with the internal channel, where the fluid conduit is connected to the inlet port. In some variations, the fluid conduit may be detachable from the inlet port. The fluid conduit may be located along an outer surface and along the length of the endoscope, and may be configured to transport fluid to the fluid delivery module separately from the endoscope. The control cable may be located along the outer surface and along the length of the endoscope. The clip may comprise an adhesive located along an endoscope-contacting surface. Alternatively or additionally, the endoscope-contacting surface of the clip may comprise an elastomeric material.

The first and second side-facing imaging elements of an imaging module may each comprise a lens assembly disposed over an image sensor, and optionally, a prism in front of each of the lens assemblies. Alternatively or additionally, each of the first and second side-facing imaging elements may comprise a prism disposed over each of the image sensors. The viewing angle for each of the first and second side-facing imaging elements may be at least 135 degrees, and/or the field of view of each of the first and second side-facing imaging elements may overlap with or be adjacent to the field of view of the front-facing imaging element of an endoscope to which the detachable imaging device is attached. In some variations, the first and second side-facing imaging elements are located co-linearly with a front-facing imaging element of the endoscope when the detachable imaging device is attached to the endoscope. In such variation, the first and second side-facing imaging elements may each comprise an image sensor and a prism (e.g., the prism may be disposed over the image sensor). Optionally, the first and second side-facing imaging elements may also comprise a lens assembly. In some variations, the first side-facing imaging element and the second side-facing imaging elements may face in opposite directions.

The fluid delivery module of a detachable imaging device may comprise a housing, where one or more portions of the housing is optically translucent. The housing of the fluid delivery module may comprise one or more curves around the first and second outlet ports that forms a fluid dynamic path toward the first and second side imaging elements for fluids exiting the first and second outlet ports. In some variations, the first outlet port is on a first side of the fluid delivery module and the second outlet port is on a second side of the fluid delivery module opposite the first side, and the internal channel may span across the fluid delivery module. For example, the internal channel may be a U-shaped cavity spanning between the first and second outlet ports.

Some variations of a detachable imaging device may further comprise a controller that is configured to combine images acquired by the first and second side-facing imaging elements and the endoscope imaging element to simulate a continuous view. The controller may be configured to stitch the images acquired by the first and second side imaging element and the endoscope imaging element into a single image having a continuous view. Optionally, the controller may be configured to output the images acquired by the first and second side-facing imaging elements and the endoscope imaging element to one or more display devices.

Another variation of a detachable imaging device for use with an endoscope having a front-facing imaging element may comprise a clip configured to be releasably disposed over a distal portion of the endoscope, an imaging module attached to the clip, and a control cable attached to the imaging module for powering and controlling the imaging module separately from the endoscope. The imaging module may comprise a top-facing imaging element having a visual axis that is perpendicular to the circumference of the clip and the visual axis of a front-facing imaging element of an endoscope, and a top-facing light source located adjacent to the first imaging element such that the light source provides illumination for the acquisition of images by the imaging element. The top-facing imaging element and the endoscope front-facing imaging element may be configured to simultaneously acquire images with different fields of view, and the field of view of the images from the top-facing imaging element may overlap with the field of view of the endoscope front-facing imaging element. The control cable may be located along an outer surface and along the length of the endoscope. In some variations, the clip may comprise an adhesive located along an endoscope-contacting surface of the clip. Alternatively or additionally, an endoscope-contacting surface of the clip may comprise an elastomeric material. In some variations, the top-facing imaging element of the imaging module may comprise a lens assembly disposed over an image sensor. Alternatively or additionally, the top-facing imaging element may comprise a prism in front of the lens assembly. In still other variations, the top-facing imaging element of an imaging module may comprise a prism disposed over the image sensor. In some variations, the viewing angle for the top-facing imaging element may be at least 135 degrees.

### BRIEF DESCRIPTION OF THE DRAWINGS

FIG. 1A is a perspective view of one variation of a secondary imaging endoscopic device disposed over an endoscope. FIGS. 1B and 1C are side-views of the secondary imaging endoscopic device and endoscope of FIG. 1A. FIG. 1D is a top view of the secondary imaging endoscopic device and endoscope of FIG. 1A. FIG. 1E is a front view of the secondary imaging endoscopic device and endoscope of FIG. 1A. FIG. 1F is an exploded view of the secondary imaging endoscopic device of FIG. 1A.

FIG. 2A is a perspective view of another variation of a secondary imaging endoscopic device disposed over an endoscope. FIGS. 2B and 2C are side-views of the secondary imaging endoscopic device and endoscope of FIG. 2A. FIG. 2D is a top view of the secondary imaging endoscopic device and endoscope of FIG. 2A. FIG. 2E is a front view of the secondary imaging endoscopic device and endoscope of FIG. 2A. FIG. 2F is an exploded view of the secondary imaging endoscopic device of FIG. 2A.

FIG. 3A is a perspective view of another variation of a secondary imaging endoscopic device disposed over an endoscope having a pivot mechanism. FIG. 3B is a top view of the secondary imaging endoscopic device and endoscope of FIG. 3A. FIG. 3C is a front view of the secondary imaging endoscopic device and endoscope of FIG. 3A. FIG. 3D is a side view of the secondary imaging endoscopic device of FIG. 3A.

FIG. 4A is a perspective view of one variation of an imaging element steering and snare extension/retraction mechanism that may be used with a secondary imaging endoscopic device. FIG. 4B is an expanded component view of the mechanism of FIG. 4A. FIG. 4C is a side-view of the mechanism of FIG. 4A. FIG. 4D is a top view of the mechanism of FIG. 4A, and FIG. 4E is a close-up of the circled region identified in FIG. 4D. FIG. 4F is a bottom view of the mechanism of FIG. 4A.

FIG. 5A is a perspective view of another variation of a secondary imaging endoscopic device disposed over an endoscope. FIGS. 5B and 5C are side-views of the secondary imaging endoscopic device and endoscope of FIG. 5A. FIG. 5D is a top view of the secondary imaging endoscopic device and endoscope of FIG. 5A. FIG. 5E is an exploded view of the secondary imaging endoscopic device of FIG. 5A. FIG. 5F is an enlarged perspective view of the secondary imaging endoscopic device (with fluid delivery module) of FIG. 5A disposed over the distal end of the endoscope.

FIGS. 6A and 6B are various perspective views of a base portion of one variation of a fluid delivery module for a secondary imaging endoscopic device. FIGS. 6C and 6D are various perspective view of an endplate portion that corresponds to the base portion of the fluid delivery module of FIGS. 6A and 6B.

FIG. 7 is a schematic representation of the fields of view and visual axes of the imaging elements of a main endoscope and one variation of a secondary imaging endoscopic device.

FIG. 8 is a cross-sectional view of one variation of tubing that may be used with a secondary imaging endoscopic device.

FIG. 9A is a schematic representation of one variation of a multi-imaging element endoscopy system; FIG. 9B is a schematic representation of another variation of a multi-imaging element endoscopy system.

FIG. 10 is a schematic representation of one variation of the layout of a display of a multi-imaging element endoscopy system.

FIG. 11A is a perspective view of one variation of a secondary imaging endoscopic device disposed over an endoscope. FIGS. 11B and 11C are side-views of the secondary imaging endoscopic device and endoscope of FIG. 11A. FIG. 11D is a top view of the secondary imaging endoscopic device and endoscope of FIG. 11A. FIG. 11E is a front view of the secondary imaging endoscopic device and endoscope of FIG. 11A. FIG. 11F is an exploded view of the secondary imaging endoscopic device of FIG. 11A.

FIG. 12A is a perspective view of another variation of a secondary imaging endoscopic device disposed over an endoscope. FIGS. 12B and 12C are side-views of the secondary imaging endoscopic device and endoscope of FIG. 12A. FIG. 12D is a top view of the secondary imaging endoscopic device and endoscope of FIG. 12A. FIG. 12E is a front view of the secondary imaging endoscopic device and endoscope of FIG. 12A. FIG. 12F is an exploded view of the secondary imaging endoscopic device of FIG. 12A.

FIG. 13A is a perspective view of one variation of a secondary imaging endoscopic device with a top-facing imaging element and corresponding light source. FIGS. 13B and 13C are side-views of the secondary imaging endoscopic device and endoscope of FIG. 13A. FIG. 13D is a top view of the secondary imaging endoscopic device and endoscope of FIG. 13A. FIG. 13E is a front view of the secondary imaging endoscopic device and endoscope of FIG. 13A. FIG. 13F is an exploded view of the secondary imaging endoscopic device of FIG. 13A with a prism disposed over the image sensor. FIG. 13G is an exploded view of the secondary imaging endoscopic device of FIG. 13A with a lens assembly disposed between the prism and the image sensor.

FIG. 14A is a perspective view of a first side of a variation of a secondary imaging endoscopic device with a single side-facing imaging element and corresponding light source. FIG. 14B is a perspective view of the second side of the secondary imaging endoscopic device of FIG. 14A. FIG. 14C is a top view of the secondary imaging endoscopic device and endoscope of FIG. 14A. FIG. 14D is a front view of the secondary imaging endoscopic device and endoscope of FIG. 14A. FIG. 14E is a side-view of the first side of the secondary imaging endoscopic device and endoscope of FIG. 14A. FIG. 14F is an exploded view of the secondary imaging endoscopic device of FIG. 14A.

FIGS. 15A-15G are perspective schematic drawings of various endoscope attachment members that may be used with any of the secondary imaging endoscopic devices described herein.

FIG. 16A schematically depicts a coordinate system based on the longitudinal axis of an endoscope to which a secondary imaging endoscopic device may be attached. FIG. 16B is a schematic representation of the fields of view and visual axes of the imaging elements of a main endoscope and another variation of a secondary imaging endoscopic device.

FIGS. 17A-17C are schematic top views of various optical system configurations that may be used in any of the imaging modules of any of the secondary imaging endoscopic devices described herein (light paths are depicted by the dotted lines)

FIG. 18A is a schematic side perspective view of another variation of a secondary imaging endoscopic device where the side-facing imaging elements are co-linear with each other and the front-facing imaging element of the main endoscope. FIG. 18B is a schematic side perspective view of the secondary imaging endoscopic device of FIG. 18A depicting the viewing angles and field of view of the front-facing and side-facing imaging elements.

### DETAILED DESCRIPTION

A secondary imaging endoscopic device may comprise an endoscope attachment member such as a sleeve that is configured to be attached over the distal portion of an endoscope, the sleeve comprising one or more side imaging elements located along the side of the elongated sleeve, and one or more light sources located along the side of the sleeve. Alternatively, a secondary imaging endoscopic device may comprise a detachable endoscope attachment member such as a sleeve or clip and an imaging module attached to the sleeve or clip. The imaging module may comprise one or more side-facing and/or top-facing imaging elements (relative to the front-facing endoscope imaging element) and one or more light sources (e.g., corresponding to each of the imaging elements). Optionally, a secondary imaging endoscopic device may also comprise a fluid delivery module attached to the endoscope attachment member and/or the imaging module, where the fluid delivery module has one or more outlet ports for delivering fluids (e.g., flush fluids, contrast fluids, therapeutic fluids, etc.). The outlet ports of the fluid delivery module may be configured to clear any debris that may accumulate on the imaging elements and/or light sources of the imaging module. Any of the secondary imaging endoscopic devices described herein may comprise an electrical conduit or control cable that extends along the length of the endoscope to which the secondary imaging endoscopic device is attached between the imaging module and a proximal controller. Such electrical conduit and/or control cable may operate separately and/or independently from the electrical conduits and/or control cables of the main endoscope. Similarly, any of the secondary imaging endoscopic devices described herein may comprise a fluid conduit that extends along the length of the endoscope to which the secondary imaging endoscopic device is attached between the fluid delivery module and a proximal fluid source. Such fluid conduit may operate separately and/or independently from the fluid conduits of the main endoscope. In some other variations, the secondary imaging endoscopic device may be coupled to a device other than another endoscope (e.g., a probe, surgical tool, etc.), and/or may be coupled to a portion other than the distal portion, e.g. a mid-portion or a proximal portion. The secondary endoscopic imaging devices described herein may be used with any endoscope (e.g., any colonoscope) as may be desired.

While the secondary imaging endoscopic devices described below are described as having one imaging module, other variations may comprise two or more imaging modules. The two or more imaging modules may have separate PCBs for each of their components, or may share a PCB (e.g. a flexible PCB). The two or more imaging modules may be connected to a proximal controller via a shared control cable, or may each have their own separate control cables. A secondary imaging endoscopic device having two or more imaging modules may have a single fluid delivery module configured to provide fluids across of the imaging modules (e.g., with multiple outlet ports located adjacent to each of the imaging modules), or may have a plurality of fluid delivery modules (e.g., one for each imaging module), as may be desirable.

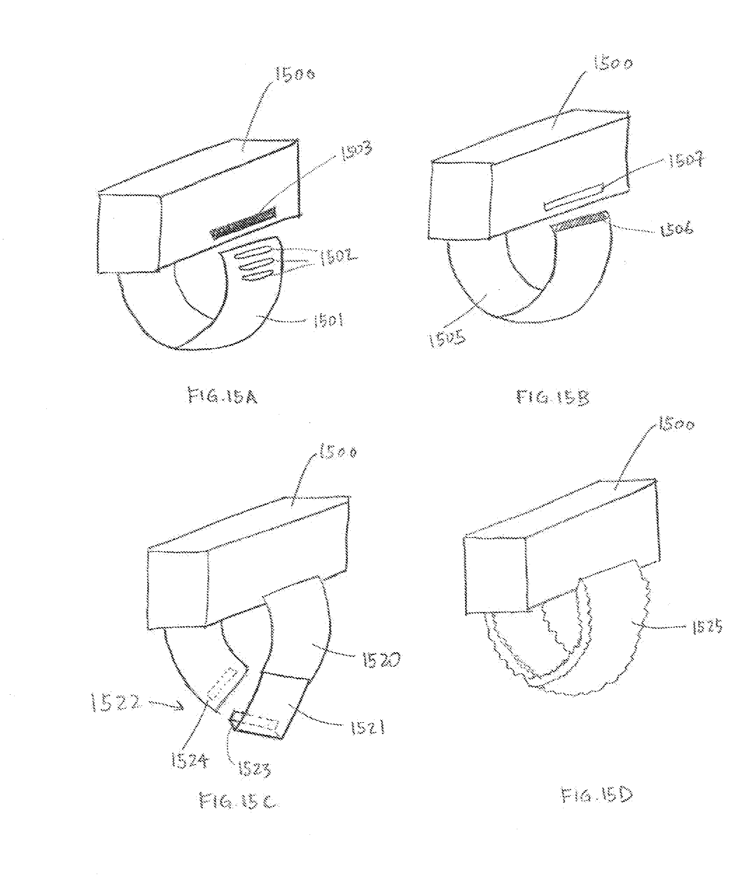

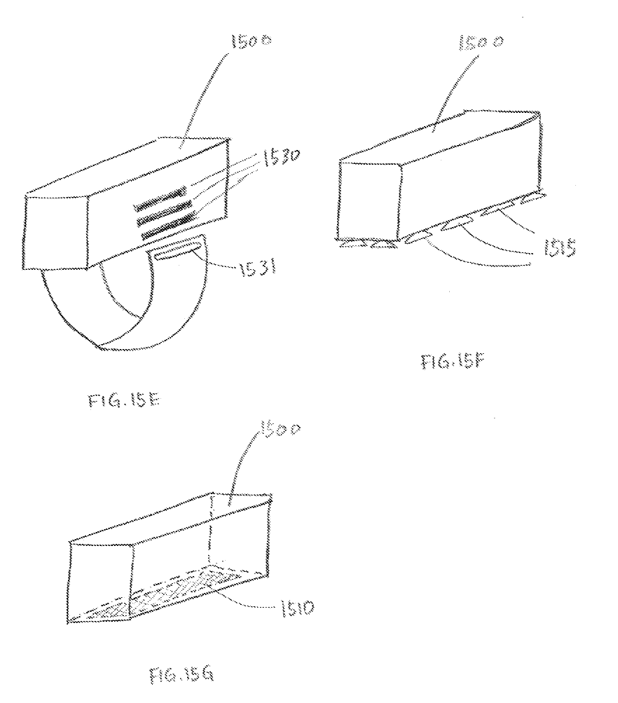

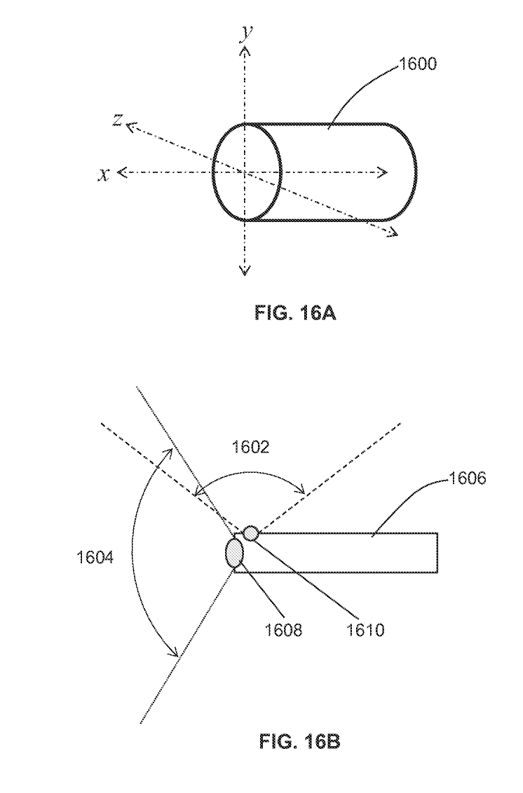

Side-facing imaging elements may provide side views (e.g., that are offset from the visual axis of the main endoscope imaging element at an angle from about 30 degrees to about 90 degrees) and/or rear views (e.g., that are about 180 degrees offset from the visual axis of the main endoscope imaging element) and/or front/antegrade views (e.g., that are in-line or parallel to the visual axis of the main endoscope imaging element), and/or rear/retrograde views (e.g., that are directly opposite or 180 degrees from the visual axis of the main endoscope imaging element). Top-facing imaging elements may provide a field of view with a visual axis that is perpendicular to the visual axes of the front-facing imaging element of the endoscope and the visual axis of a side-facing imaging element (if any), and may also provide side-views and rear-views. That is, within an x-y-z coordinate system (FIG. 16A), the visual axis of the front-facing imaging element of the endo scope may be parallel to (or at an angle less than 90 degrees from) the x-axis, the visual axis of a side-facing imaging element may be parallel to (or at an angle less than 90 degrees from) the z-axis, and the visual axis of a top-facing imaging element may be parallel to (or at an angle less than 90 degrees from) the y-axis. In some variations, the locations of the side-facing imaging elements may not be co-linear or aligned with the location of the front-facing imaging element (i.e., the location of the front-facing imaging element may have a different y-axis value than the side-facing imaging elements, or the side-facing imaging elements may be located above the front-facing imaging element). In other variations, the location of the front-facing and side-facing imaging elements may be aligned or co-linear such that they form a line parallel with the z-axis (i.e., have the same y-axis value). Aligning the front-facing and side-facing imaging elements in such configuration may facilitate the stitching of images acquired by the front-facing and side-facing imaging elements.

The endoscope attachment member of a secondary imaging endoscopic device may comprise a sleeve with a distal lip that is configured to engage a distal edge of the endoscope so that the sleeve remains securely attached to the endoscope during use. Additionally or alternatively, the sleeve may comprise a proximal ridge or protrusion that may engage a sidewall of the endoscope such that the secondary imaging endoscopic device is retained over the endoscope. In some variations, the sleeve of the secondary imaging endoscopic device may be attached to the endoscope by friction-fit, screw-fit, compression-fit, etc. FIGS. 15A-15G depict various releasable endoscope attachment members that may be used with any of the secondary imaging endoscopic devices described herein. The imaging module and/or fluid delivery module is schematically represented by module 1500, which may have any of the form factors described in detail below. Imaging and fluid delivery modules may be integrally formed, or may be separate components that are coupled together (either permanently or releasably). Module 1500 may represent an imaging module only, a fluid delivery module only, or the combination of the imaging and fluid delivery modules. In some variations, an add-on secondary imaging endoscopic device may comprise a releasable endoscope attachment member such as a clip, band, or strap configured to attach the secondary imaging endoscopic device to the distal portion of an endoscope (e.g., by friction-fit, screw-fit, compression-fit, etc.), while in other variations, an imaging module and/or fluid delivery module may comprise attachment members on their housing without the use of a clip, band or strap. In some variations, the clip, band, strap or sleeve may be at least partially made of an elastomeric material, such as silicone or latex. As depicted in FIG. 15A, a strap 1501 may have protrusions 1502 or beads (e.g., a string of beads) that are configured to interfit with a receptacle 1503 (e.g., one or more notches, grooves, ridges, recesses, etc.) located on the module 1500. For example, the strap attachment mechanism may be similar to the ratchet mechanism of a cable or zip tie. As depicted in FIG. 15E, a module 1500 may comprise one or more receptacles or notches 1530 in different places such that articulating (e.g., snap-fitting) the protrusions 1531 or beads with different receptacles allows the attachment member to secure to endoscopes of various sizes. In still other variations, an add-on secondary imaging endoscopic device may comprise a strap 1505 with one or more magnetic components 1506 (e.g., rare earth magnets) that attach to a magnetic material 1507 located on the module 1500. Such magnetic attachment may secure (e.g., tighten) the strap 1505 around the endoscope and/or may act to attach the module 1500 to the strap 1505. The endoscope attachment member or the imaging and/or fluid delivery module may also comprise an adhesive portion or region of increased friction along the endoscope-contacting surface. Examples may include various glues, tacky coatings or films (e.g., double-sided adhesive tape), rubber or silicone-based materials and the like. For example, FIG. 15G depicts a module 1500 having a region 1510 that may comprise an adhesive or any of the high-friction materials described above, without a strap or clip. Alternatively or additionally, the endoscope-contacting surface of an endoscope attachment member or imaging and/or fluid delivery module may also comprise one or more micro-suction cups. FIG. 15F depicts module 1500 with a plurality of micro-suction cups, without a strap or clip. In some variations, endoscope attachment members may provide a releasable attachment to the endoscope by compression-fit mechanisms. FIG. 15C depicts one example of an endoscope attachment member that may include a semi-circular ring 1520 or clip with a hinged arm 1521 or clamp that cinches the ring or clip around the endoscope when engaged with a locking mechanism 1522 (e.g., a flip-to-lock mechanism that includes a lip 1523 on the hinged arm and a recess 1524 on the opposite segment of the ring). Alternatively or additionally, a releasable endoscope engagement member may comprise an air bladder 1525 (e.g., in a ring-shape, or semi-circular shape) that tightens over the endoscope as it is inflated, as schematically depicted in FIG. 15D. Kits may be provided where the secondary imaging endoscopic device comprises an imaging module, a fluid delivery module and a plurality of endoscope attachment members configured to releasably attach to the imaging and/or fluid delivery modules. The endoscope attachment members may each be sized and shaped to attach to endoscopes of various sizes, shapes, materials, etc. Once a particular attachment member has been selected for a particular endoscope, the imaging and/or fluid delivery modules may be attached to the attachment member (e.g., by snap-fit, screw-fit, etc.) and installed over the endoscope. Such kit may enable a practitioner to customize the attachment and/or fit of the secondary imaging endoscopic device to a particular endoscope of his or her choosing. In still other variations, the imaging mechanisms (e.g., side imaging elements and light sources) described herein may be integrated into the distal end of an endoscope, such that a separate add-on device is not required.

In still other embodiments, the primary endoscope and secondary imaging endoscopic device may both be configured to form a mechanical interfit. For example, the primary endoscope and/or secondary endoscopic device may comprise one or more recesses, slots or grooves configured to receive a protruding structure on the other endo scope.

The side imaging element(s) of a secondary imaging endoscopic device may be located along the circumference of the endo scope engagement member (e.g., sleeve or clip), and/or located in an imaging module attached to the endoscope attachment member. For example, a secondary imaging endoscopic device may comprise a first side imaging element at a first circumferential location on the side of the sleeve, and a second side imaging element at a second circumferential location on the side of the sleeve that is 180 degrees from the first location. In other examples, any two of the side imaging elements may be no more than (or at least) 45 degrees apart, no more than (or at least) 90 degrees apart, or no more than (or at least) 120 degrees apart. Each side imaging element may comprise an image sensor, and the side imaging elements may be oriented such that the field of view of each side imaging element may be offset from the longitudinal axis of the endoscope (or lumen of the sleeve). For example, the visual axis of a side imaging element may be tangential to a circumference of the sleeve, or may be perpendicular to the circumference of the sleeve. In other variations, the visual axis of a side imaging element may be skewed relative to the longitudinal axis of the endoscope, e.g. comprising a non-parallel, non-intersecting configuration, or a non-coplanar configuration with the longitudinal axis of the endoscope. In some variations, the visual axis of a side imaging element may be at an angle with respect to the visual axis of the main imaging element of an endoscope. For example, the visual axis of a side imaging element may be about 45 degrees, about 90 degrees, about 135 degrees, etc. from the visual axis of the main endoscope imaging element. In some variations, the visual fields of the main and side imaging elements may overlap with each other. For example, the angular spread of the overlap between the visual fields of the main and side imaging elements may be from about 15 degrees to about 70 degrees, e.g., about 25 degrees, about 30 degrees, about 45 degrees, about 60 degrees. The visual axes of the side imaging elements may be aligned (e.g., co-linear and co-planar), angled (e.g., co-planar), and/or askew (e.g., not co-planar) with respect to each other. For example, the visual axis of a first side imaging element may be tangential to the circumference of the sleeve, and the visual axis of a second side imaging element opposite to the first side imaging element (e.g., 180 degrees away from the first imaging element) may also be tangential to the circumference of the sleeve. Additional details regarding the field of view and/or visual axis of various embodiments of an endoscope and/or secondary imaging endoscopic device are described below. It should be noted that although the examples of secondary imaging endoscopic devices described herein have two side imaging elements, it should be understood that a secondary imaging device may have more than two side imaging elements (e.g., 3, 4, 5, 6, 8, 10, 12, etc. imaging elements) as may be desired. Two or more side imaging elements may be helpful for acquiring sufficient image data for reconstructing a 360 degree view (i.e., three-dimensional volume) of a body cavity of interest.

The side-facing imaging elements of an imaging module may optionally comprise a movement mechanism that allows the visual field of the one or more side-facing imaging elements to be adjusted. The movement mechanism may allow the side imaging elements to pivot between proximal and distal positions (e.g., parallel to the longitudinal axis of the endoscope) and/or to translate along a circumference of the sleeve. In some variations, the side imaging element may be retractable. The orientation of a side imaging element may be adjusted depending on the distance from the side imaging element to the side wall of the body cavity. For example, the imaging element orientation of a first side imaging element of a secondary imaging endoscopic device and the imaging element orientation of a second side imaging element of the secondary imaging endoscopic device may be adjusted such that the overlap in their visual fields of view remains constant. A controller in communication with the side imaging elements of a secondary imaging endoscopic device may be able to detect whether a tissue wall is drawing closer to the side imaging elements, and pivot the side imaging elements such that the field of view sweeps away from the tissue wall. The visual axes of the side imaging elements may also be adjusted so that the image overlap between the imaging elements (e.g., between each side imaging element and the endoscope imaging element) is kept at a consistent value. For example, the side imaging elements may be pivoted such that the overlap between the images acquired by the side imaging elements and/or the endoscope imaging element is kept at up to 1%, 5%, 10%, 15%, 20%, 30%, 45%, etc.

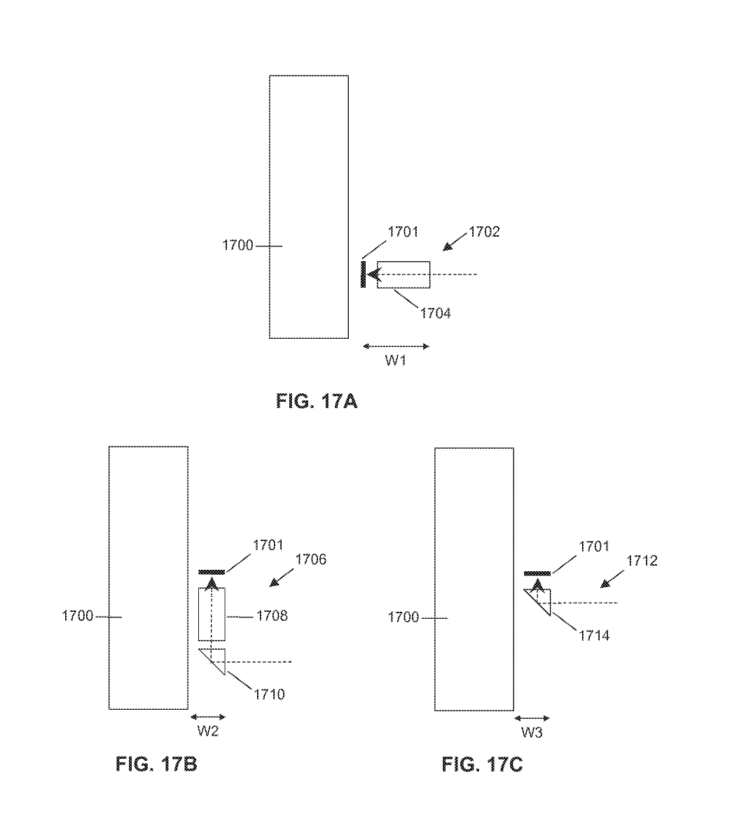

Optionally, the side imaging elements of a secondary imaging endoscopic device may comprise a filter, e.g., an infrared filter, which may help to enhance the acquired image. An infrared filter may result in image that facilitates detection of an adenoma. In some variations, a polarizing filter or film, e.g. a Dual Brightness Enhancement Film (DBEF) by 3M™ (St. Paul, Minn.) may be located over the image sensor of a side imaging element, which may enhance the acquired image by improving the contract or brightness of the image. Alternatively or additionally, the side imaging element(s) may comprise a bandpass filter. For example, a side imaging element may comprise a bandpass filter that allows for the transmission of light having a wavelength between about 445 nm to about 500 nm, e.g., about 450 nm to about 490 nm. A bandpass filter with such transmission characteristics may be used in a system where the corresponding side light sources emit light in the green-blue spectrum (e.g., about 445 nm to about 500 nm), which may allow for the visualization of deeper tissue structures and/or features (e.g., beneath a mucosal layer). The side imaging element(s) of the secondary imaging endoscopic device may comprise a high definition image sensor (e.g., HD CMOS, CCD) or a standard definition image sensor. In some variations, the image sensor may have a high dynamic range to adequately image both high light and low light regions without over- and/or under-saturating the sensor. Optionally, the side imaging element(s) may have a cover or cap over the image sensor, lens and/or other optical components, which may help to shield the optical components from debris and fluids. The focal depth of the lens may be from about 1 mm to about 150 mm, e.g., about 2 mm to about 45 mm. In some variations, the one or more side-facing imaging elements may comprise a lens assembly disposed over the image sensor to focus the light before it impinges on the image sensor. A prism (with or without a wavelength filter, such as an infrared filter) may be disposed over the lens assembly so that the optical path of the light re-directed towards the lens assembly. Such configuration may be used when a light path that is parallel to the longitudinal axis of the endoscope is desired. Alternatively or additionally, a prism may be included in the light path in order to filter and/or magnify and/or focus this image before it passes through the lens assembly to the image sensor. In some variations, the one or more side-facing imaging elements may not comprise a lens assembly, but may have a prism that filters and/or magnifies and/or focuses the light before it impinges on the image sensor. The optical path and associated optical components in an imaging module may be selected such that the overall size and profile of the imaging module is reduced. For example, an optical component that performs two functions (e.g., filters and focuses light) may be selected in place of two optical components that each perform a different function. In some variations, directing a light path in a particular orientation may help to reduce the width and/or height of the secondary endoscopic imaging device. Various optical paths and components that may be used with any of the secondary endoscopic imaging devices described herein are depicted in FIGS. 17A-17C. Although FIGS. 17A-17B are top views of an endoscope and exemplary optical systems located on the side of the endoscope, it should be understood that any of the depicted optical systems may be located on the top, bottom, or any other location along the circumference of the endoscope. FIG. 17A depicts an endoscope 1700 and the optical system 1702 of one variation of a secondary endoscopic imaging device comprising an image sensor 1701 (e.g., a CCD or CMOS sensor) and a lens assembly 1704. The light path is depicted by the dotted line. As shown, the light path is perpendicular to the longitudinal axis of the endoscope 1700. Such configuration may have a width W1 from about 5 mm to about 8 mm, which may largely comprise the length of the lens assembly 1704 (which may be longer than it is wide, which may be from about 5 mm to about 7 mm). FIG. 17B schematically depicts another variation of an optical system 1706 of a secondary endoscopic imaging device comprising an image sensor 1701, a lens assembly 1708 and a prism 1710. The prism 1710 introduces a bend in the light path (e.g., a 90 degree turn) such that the lens assembly 1708 may have a parallel orientation with respect to the endoscope. Such configuration may have a width W2 from about 2 mm to about 5 mm, which may largely comprise of the width of the prism and/or lens assembly and/or image sensor (which may both be less than the length of the lens assembly). A prism may have a width from about 1.5 mm to about 3 mm, an image sensor (such as a CMOS or CCD sensor) may be about 1.8 mm by 1.8 mm (with a diagonal of about 2.6 mm), and a lens assembly may have a width from about 2 mm to about 4 mm, e.g., about 2.8 mm. FIG. 17C schematically depicts another variation of an optical system 1712 of a secondary endoscopic imaging device comprising an image sensor 1701 and a prism 1714. In this variation, the prism 1714 not only bends the light path, but also magnifies and focuses the light prior to impinging on the image sensor 1701. Such configuration may have a width W3 from about 2 mm to about 5 mm, which may largely comprise of the width of the prism (which may be less than the length of a lens assembly that performs similar levels of magnification and/or focusing). In some variations, widths W2 and W3 may be the same. Bending the light path with a prism may help the secondary endoscopic imaging device to have a smaller profile. For example, a secondary endoscopic imaging device having two side-facing imaging elements having the configuration of FIG. 17B or 17C may have an overall width from about 4 mm to about 10 mm, as compared to a secondary endoscopic imaging device having two side-facing imaging elements having the configuration of FIG. 17A, which may have an overall width from about 10 mm to about 14 mm. Optionally, one or more optical filters (e.g., an IR, near-IR, UV, or any wavelength filter or polarizing filter) may be provided with the optical systems of FIGS. 17A-17C. The optical filter may be a separate component, or may be integrated with the lens assembly and/or the prism. Although the various embodiments of secondary endoscopic imaging devices described in detail below may comprise optical systems having one of the optical systems depicted in FIGS. 17A-17C, it should be understood that such embodiment may alternatively comprise any of the other optical systems, as may be desirable.

A secondary imaging endoscopic device may comprise one or more light sources that illuminate the field of view of each of the side imaging elements. For example, each side imaging element of a secondary imaging endoscopic device may have a corresponding light source adjacent to it. A secondary imaging endoscopic device light source may radiate visible and/or infrared light. Alternatively or additionally, a secondary imaging endoscopic device light source may radiate light of any desired wavelength, including green, blue, white (e.g., broadband) and/or UV light. In some variations, a secondary imaging endoscopic device may have a single LED light source that emits visible light, while in other variations, a secondary imaging endoscopic device may have a first LED light source that emits visible light and a second LED light source that emits infrared light. Alternatively or additionally, a side light source may emit light having wavelengths in the green-blue spectrum (e.g., from about 445 nm to about 500 nm). Illumination of tissue by green-blue light may allow for the visualization and imaging of deeper tissue features and/or structures. For example, green-blue light may be capable of penetrating through mucosal layers so that features beneath a mucosal layer may be examined by a practitioner. Optionally, a filter (e.g., a bandpass filter) or polarizer may be located over the light source, for example, DBEF or an infrared filter. In some variations, a side light source may emit light across a broad spectrum, and the imaging element may comprise a filter or polarizer that selectively transmits certain light of certain wavelength and/or orientation for capture by the imaging sensor.

A secondary imaging endoscopic device may also comprise an accelerometer, force sensor, pressure sensor, and/or a position sensor, or other type of tracking mechanism. Such sensors may provide feedback to a practitioner to facilitate steering the endoscope to which a secondary imaging endoscopic device is attached. An accelerometer may measure the motion and direction of the distal portion of the endoscope. This may help to inform a practitioner of any abrupt or discontinuous orientation changes in the endoscope position, which may indicate that the endoscope is looping or forming kinks as it is advanced within a tubular cavity. For example, an accelerometer on a secondary imaging endoscopic device attached to a colonoscope may help prevent looping during intubation, and may also provide information to a practitioner as to whether an area of the colon has already been imaged or still needs to be imaged. For example, a controller may use data from the accelerometer in an algorithm to generate an indicator on a virtual map of a colon to mark areas in the colon for which an image has been acquired. A force sensor and/or pressure sensor may provide tactile feedback to the practitioner as the endoscope is steered within the patient, which may help to reduce discomfort to the patient during the procedure.

A control cable or electrical conduit may connect the imaging module of a secondary imaging endoscopic device to a controller located at a proximal portion of the secondary imaging endoscopic device. The control cable or electrical conduit may extend along an outside surface of (e.g., external to) the endoscope, between the imaging module and the proximal controller. The cable may be secured to the endoscope with clips and the like, or may not be secured to the endoscope beyond its attachment via the secondary imaging endoscopic device. The control cable may comprise wires and/or flexible PCBs that power the secondary imaging endoscopic device separately and/or independently from the endoscope, and may also comprise wires and/or buses that allow a proximal controller to control the imaging module separately from the endoscope. For example, the control cable may turn on or off the side-facing imaging elements and/or adjust the intensity of the corresponding light sources regardless of whether the endoscope imaging element(s) and/or light source(s) are turned on. In some variations, a secondary endoscopic imaging device may have a plurality of control cables or electrical conduits, as may be desirable. For example, a secondary endoscopic imaging device having two side-facing imaging elements that are on opposite sides from each other may have two separate control cables or electrical conduits, each separately connected to a side-facing imaging element. Alternatively, a plurality of side-facing imaging elements and/or imaging modules may share the same wiring and/or PCBs such that there is only one cable or electrical conduit extending between the plurality of imaging elements and/or imaging modules and a proximal controller.

Surgical tools, such as a biopsy tool, a snare or forceps, may be advanced through a working lumen in the endoscope. Alternatively or additionally, a secondary imaging endoscopic system may comprise its own lumen, separate from the working lumen of the main endoscope, through which such tools may be advanced. Irrigation fluids and the like may also be provided to the body cavity via the endoscope and/or secondary imaging endoscopic device lumens. In some variations, the secondary imaging endoscopic system may comprise a separate irrigation channel that may be used to deliver a cleaning fluid to the distal portion of the endoscope and/or secondary imaging endoscopic device to clean the lens of the side imaging elements and/or imaging sensors. Any of the variations described herein may comprise one or more ports for fluid infusion and/or the delivery of surgical instruments.

Some variations of a secondary endoscopic device may comprise one or more channels or ports for the passage of fluids therethrough. For example, a secondary imaging endoscopic device may comprise a fluid delivery module that has one or more fluid delivery/outlet ports provided in the proximity of the side imaging elements so that a solution (e.g., saline) or air may be delivered to the imaging elements for clearing away visual obstructions. The fluid conduit that connects the fluid delivery module to a proximal fluid source may be located along an external length of the endoscope, and may optionally be secured to the endoscope via clips (e.g., may be similar to the control cable). The fluid conduit may be separate from any fluid conduits in the endoscope. For example, fluid may be transported through the fluid conduit to the fluid delivery modules of the secondary imaging endoscopic device without transporting fluid through the endoscope (and vice versa). This may provide the capability to clear off debris from just the side-facing imaging elements or just the front-facing imaging element (i.e., the endoscope imaging element). The fluid delivery module may comprise an inlet to which the fluid conduit is attached. The inlet may connect to an internal fluid conduit or channel that is in communication with one or more outlet ports. The number of fluid outlet ports may correspond to the number of side-facing imaging elements. There may be one or more curves along the surface of the secondary endoscopic device housing near the fluid outlet port that may help to direct the fluid exiting the port towards the side imaging elements and/or light sources. For example, the curvature of the image module and/or fluid delivery module housing near the fluid delivery ports may encourage fluid flow towards the optical components of the secondary endoscopic device (e.g., the side imaging element, light source, etc.) while impeding fluid from flowing or moving across non-optical portions of the device. The curvature around and adjacent to the imaging elements may help to facilitate fluid flow from the port(s) across the imaging elements and then away from the imaging elements. For example, a flush fluid (e.g., saline) may exit the port, sweep across the adjacent imaging element, then sweep away from the imaging element. Providing a streamlined, fluid-dynamic path from the port to the optical components and then away from the optical components may help to clear or remove any debris obscuring or interfering with the imaging process.

The one or more fluid delivery channels or ports may be integrally formed with the sleeve of the secondary imaging endoscopic device or the main imaging device (e.g., endoscope), or may be embodied in a separate module that is attachable to the sleeve of the secondary imaging endoscopic device. In some variations, a fluid delivery module may have one or more optically transparent or translucent portions so that it does not interfere substantially with the function of the optical components (e.g., allows light from the light source to pass through with little or no attenuation, and/or light to pass through to the side imaging elements). Alternatively, the fluid delivery module may be attached to the endoscope attachment member (e.g., sleeve or clip) and/or housing and/or imaging module such that it does not cover all or any of the optical components. For example, when attached to the endoscope attachment member, the fluid delivery module may not cover the side-facing imaging devices, but a translucent or transparent portion of the module may cover the light sources. The fluid delivery module may be attached to the endoscope attachment member of the secondary imaging endoscopic device by friction-fit, snap-fit, compression-fit, and/or may be attached using screws, adhesives, magnets, etc. The housing of the fluid delivery module may be made of any suitable polymer, such as polycarbonate (with varying opacity), polyetherimide, etc.

The endoscope and/or secondary imaging endoscopic device may be in communication with a controller that is configured to store and process acquired images and video, receive signals from the accelerometer, pressure sensor, and/or force sensor, as well as to steer the endoscope and control the orientation of the side imaging elements. The communication may be wired or wireless or a combination of both. The controller may be pre-programmed with an algorithm that correlates the collected image and/or video data with data from the accelerometer and/or position sensors. Such data may be used in an image processing algorithm for combining the images acquired from the secondary imaging endoscopic device side imaging elements and the endoscope imaging element. For example, positional and/or accelerometer data may be used to locate in three-dimensional space the location where an image was acquired, so that if a practitioner needs to return to that location (e.g., to contact a previously imaged polyp), s/he may do so based on the positional and/or accelerometer data associated with that particular image or video. The controller may also be pre-programmed with an algorithm for stitching the images from the side imaging elements of the secondary imaging endoscopic device and the main endoscope imaging element, to provide a continuous view. For example, the images may be stitched to create a continuous 180 degree or 360 degree view of the body cavity. Alternatively or additionally, the controller may output the images from the secondary imaging endoscopic device side imaging elements and endoscope imaging element to one or more display devices (e.g., a monitor). For example, all the images acquired from all the imaging elements may be displayed on one display device, and arranged to simulate a continuous view. In some variations, the image from the endoscope imaging element may be presented as a front view, while the images from the secondary imaging endoscopic device side imaging elements may be stitched together to form a continuous view of the side of the body cavity. Images and/or video acquired from side imaging elements may be scaled and/or cropped to match the aspect ratio of the images and/or video acquired from the main endoscope imaging element. The controller may be connected to the secondary imaging endoscopic device by one or more wires, or may be wirelessly connected.

Optionally, a system comprising an endoscope and/or secondary imaging endoscopic device may comprise a controller having one or more video processors configured to analyze and/or store the images acquired by the various imaging elements and one or more monitors or displays. In some variations, the video processor(s) managing the data from the side imaging elements may be synchronized with the video processor(s) managing the data from the main endoscope imaging device. An endoscopic system may also comprise a data relay that may assemble the image data from various imaging elements and/or video processors, as well as physiological data (e.g., vital data including heart rate, breath rate, blood pressure, etc.) from various devices for display on the one or more monitors.

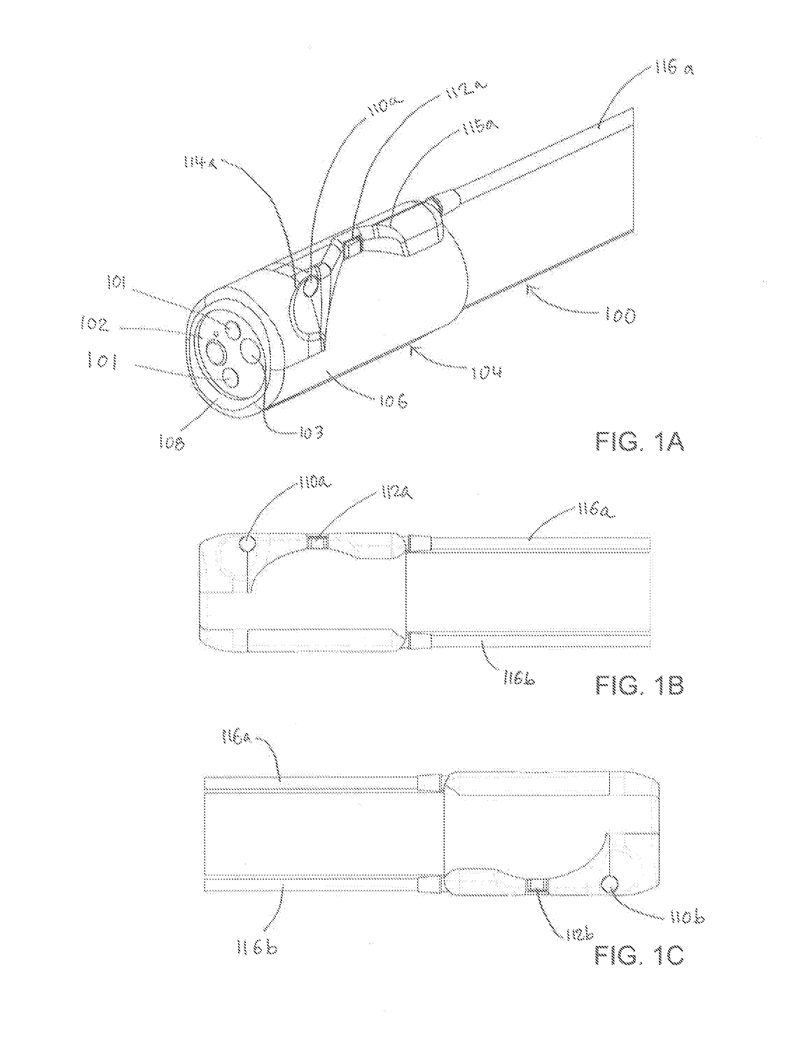

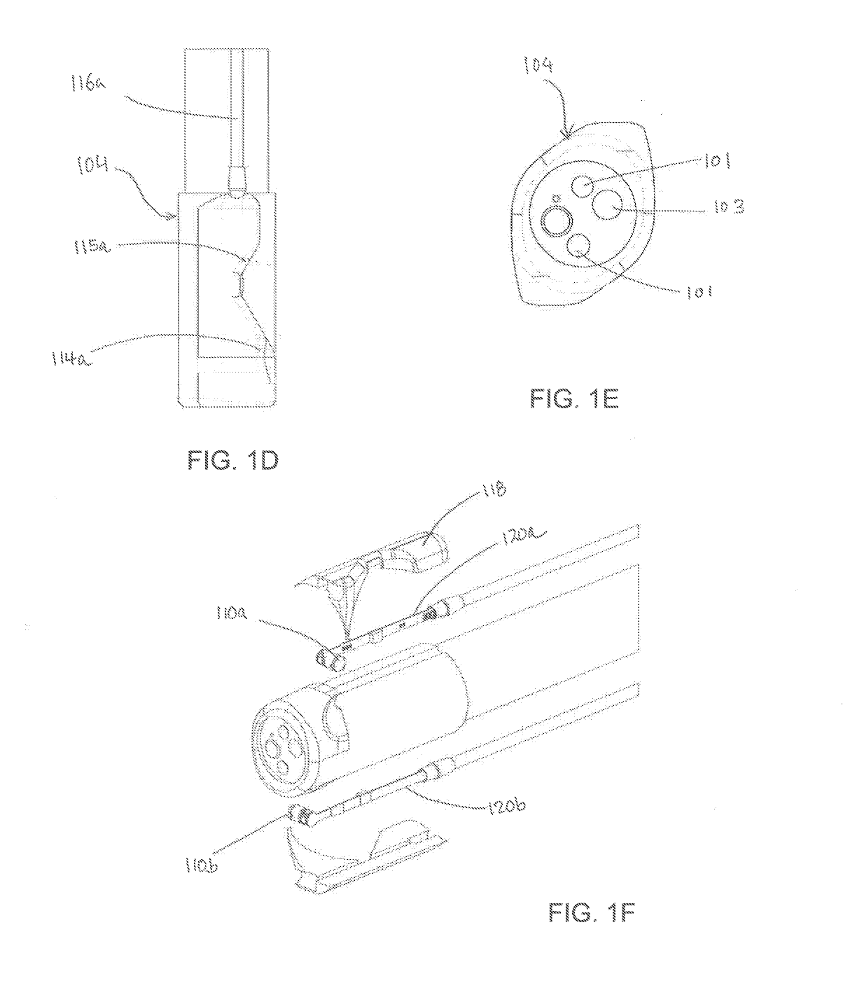

One example of a secondary imaging endoscopic device 104 disposed over a distal portion of an endoscope 100 is depicted in FIGS. 1A-1F. The secondary imaging endoscopic device 104 may comprise a sleeve 106, a first side-facing imaging element 110a, a first light source 112a, a second side-facing imaging element 110b and a second light source 112b, where the first and second side imaging elements and first and second light sources are attached to the sleeve at different circumferential locations. The side-facing imaging elements 110a,b may comprise any of the optical components and may be configured according to any of optical systems described and depicted in FIGS. 17A-17C. In this variation, the first side imaging element 110a and first light source 112a are located about 180 degrees from the second side imaging element 110b and second light source 112b around the circumference of the sleeve 106, but in other variations, the first side imaging element and light source may be located 30 degrees, 60 degrees, 90 degrees, 120 degrees, 150 degrees, etc. from the second side imaging element and light source. Each side imaging element and light source pair faces in the same direction, such that the light source provides illumination for the imaging element. The side imaging elements and light sources face in a direction that is tangential to a circumference of the sleeve 106 such that light radiated from the light sources is directed across the outer surface of the sleeve 106 (e.g., the direction of the illuminated light forms a 0 degree angle with respect to the surface of the sleeve). For example, the visual axes of the side imaging elements may be tangential to the circumference of the sleeve. Optionally, the orientation (and therefore the visual axes) of the side imaging elements 110a,b and light sources 112a,b may adjusted by a displacement mechanism (such as any of the mechanisms described below). For example, the visual axes of the side imaging elements may be perpendicular to the outer surface of the sleeve 106 such that light radiated from the light sources is directed away the outer surface of the sleeve 106. The visual axis of a side imaging element may form an angle with respect to the surface of the sleeve that may vary between about 0 degrees and about 180 degrees, for example, the angle may be about 0 degrees, about 30 degrees, about 45 degrees, about 60 degrees, about 90 degrees, about 120 degrees, about 150 degrees, about 170 degrees, etc. The side imaging elements and light sources may be located within one more recesses along the outer surface of the sleeve 106. For example, the side imaging element 110a may be located within a recess 114a (and the side imaging element 110b may be located in a corresponding recess 114b that is located 180 degrees from the recess 114a). The curvature of the recesses 114a,b may be such that the field of view of the side imaging elements 110a, b are not impeded. For example, the curvature of the recesses 114a,b may permit the side imaging elements 110a,b to have a viewing angle of about 90 degrees, about 100 degrees, about 120 degrees, about 135 degrees, about 180 degrees, etc. The recesses 114a,b may also shield the side imaging elements 110a,b from the endoscope light sources (e.g., light sources 101), so that the endoscope light source does not interfere with (e.g., by oversaturating) the image sensors of the side imaging elements 110a,b. The light sources 112a,b may be located in recesses 115a,b that are adjacent to recesses 114a,b, respectively, which may help direct the light radiated from the light sources to illuminate the field of view of the corresponding side imaging elements. The light sources 112a,b may be any suitable light source, including LEDs and the like. Optionally, filters may be disposed over the side imaging elements and/or the light sources, for example, infrared filters, DBEF polarizers, neutral density filters, etc. In some variations, filters of the same or different type may be disposed over both the side imaging element and the light source, while in other variations, there may only be a filter disposed over either the side imaging element or the light source. Alternatively or additionally, there may be a clear lens disposed over the side imaging elements and/or the light sources that may act to protect the underlying optical structures from tissue fluids. The sleeve may be made of any suitable material, including polymers such as polyetherimide (e.g., ULTEM™ 1000), polycarbonate, and the like.

The secondary imaging endoscopic device 104 may have a distal lip 108 that is configured to engage with the distal end of the endoscope 100. Optionally, a pressure and/or force sensor may be located on the distal lip 108 so that the contact force between the distal-most edge of the secondary imaging endoscopic device 104 and the wall of the body cavity may be measured and conveyed to the controller and/or practitioner. Optionally, the secondary imaging endoscopic device 104 may also comprise an accelerometer 118 located on the sleeve 106. The data from the accelerometer 118 and/or the pressure sensor and/or force sensor may be communicated wirelessly to a controller at a proximal end, or a separate controller. Alternatively, the data from these sensors, along with the image data from the side imaging elements may also be communicated to a proximal controller via an electrical conduit 120 enclosed within an elongate tube or catheter 116, as depicted in FIG. 1F. The electrical conduit 120 may also convey signals from the controller that operate the pivot mechanism of the side imaging elements and/or light source, adjust the intensity of the light sources, activate or deactivate the side imaging elements and/or light sources, etc. There may be a first control cable or tube 116a that encloses the electrical communication conduits between the first side imaging element 110a and light source 112a, and a second control cable or tube 116b that encloses the electrical communication conduits between the second side imaging element 110b and light source 112b. The electrical conduits 120a,b may be, for example, one or more wires and/or a flexible circuit board, that extend along the length of the tubes 116a,b. The control cables or tubes 116a,b may optionally comprise additional longitudinal channels or lumens for surgical tools and/or fluid infusion. For example, the tubes or catheters may comprise a biopsy channel and/or an irrigation channel. Alternatively or additionally, surgical tools and/or irrigation fluids may also be provided through one or more working lumens 103 of the endoscope 100. The proximal controller may control the operation of these surgical tools and/or fluid irrigation via the electrical conduits 120a,b. The electrical conduits and/or control cables of a secondary endoscopic imaging device may be separate and/or independent from the electrical conduits and/or control cables of the main endoscope. Although the control cables or tubes depicted here extend longitudinally along an external surface of the endoscope, in other variations, the control cables or tubes may be located within a lumen of the main endoscope and/or be embedded within the main endoscope.

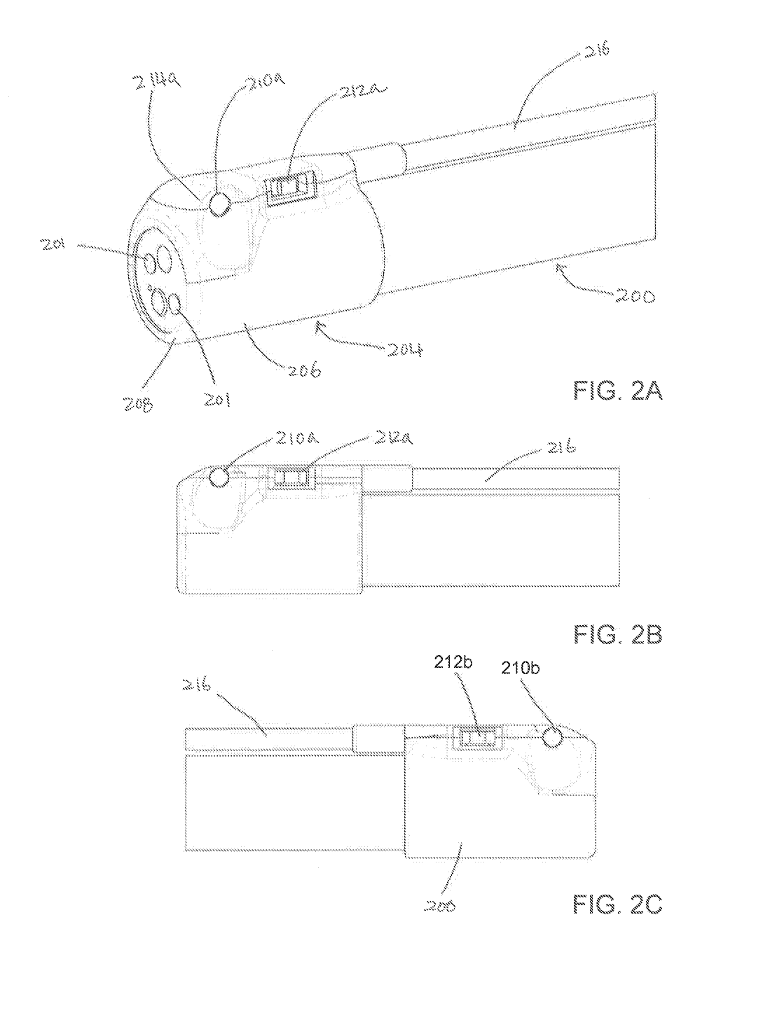

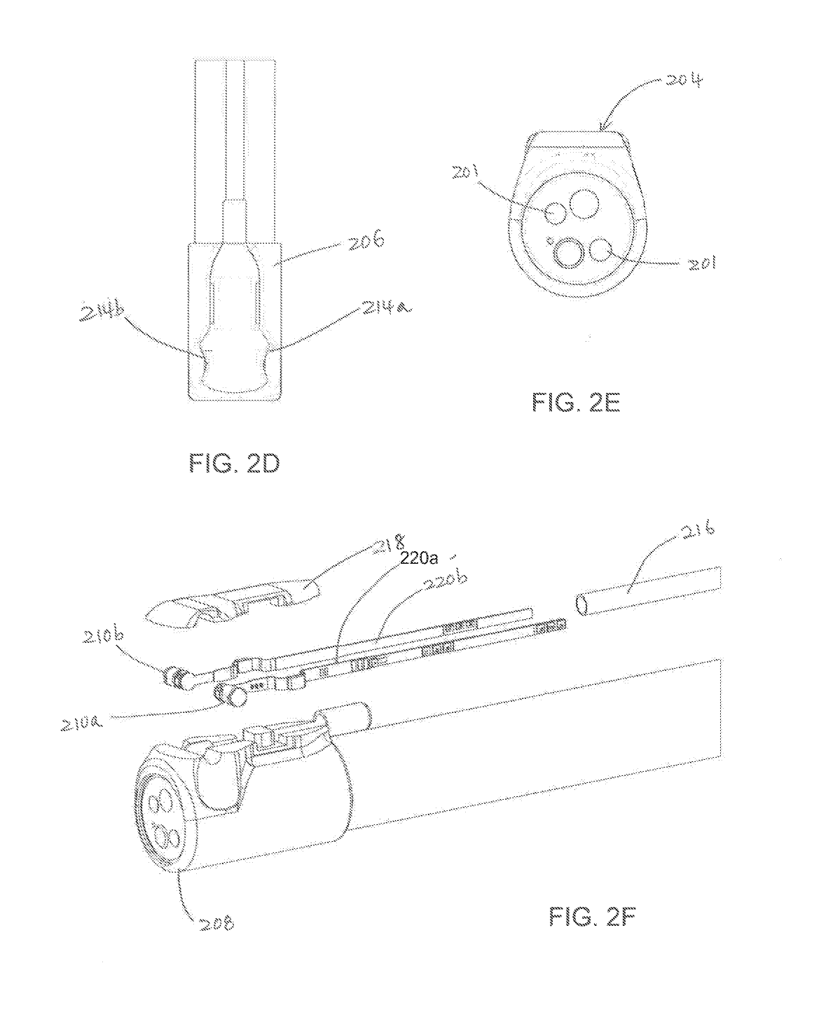

Another example of a secondary imaging endoscopic device 204 disposed over a distal portion of an endoscope 200 is depicted in FIGS. 2A-2F. The side imaging elements and light sources of the secondary imaging endoscopic device 204 may be located adjacently about the same circumferential location around the sleeve of the secondary imaging endoscopic device 204, but face in opposite directions. The secondary imaging endoscopic device 204 may comprise a sleeve 206, a first side-facing imaging element 210a, a first light source 212a, a second side-facing imaging element 210b and a second light source 212b, where the first and second side imaging elements and first and second light sources are attached to the sleeve such that the visual axes of the side imaging elements form a line that is tangential to a circumference of the sleeve 206. As described above, each side imaging element and light source pair faces in the same direction such that the light source provides illumination for the side imaging element. The side-facing imaging elements 210a,b may comprise any of the optical components and may be configured according to any of optical systems described and depicted in FIGS. 17A-17C. The visual axes of the side imaging elements and light sources may be tangential to the circumference of the sleeve 206 such that light radiated from the light sources is directed across the outer surface of the sleeve 206 (e.g., the direction of the illuminated light forms a 0 degree angle with respect to the circumference of the sleeve). Optionally, the orientation of the side imaging elements 210a,b and light sources 212a,b may adjusted by a pivot mechanism (such as the ones described below). The side imaging elements and light sources may be located within one more recesses along the outer surface of the sleeve 206. For example, the side imaging element 210a may be located within a recess 214a (and the side imaging element 210b may be located in a corresponding recess 214b that is opposite the recess 214a, as depicted in FIG. 2D). The curvature of the recesses 214a,b may be such that the fields of view of the side imaging elements 210a, b are not impeded. For example, the curvature of the recesses 214a,b may permit the side imaging elements 210a, b to have a viewing angle of about 90 degrees, about 100 degrees, about 120 degrees, about 135 degrees, about 180 degrees, etc. and may provide some shielding from the endoscope light sources (e.g., light sources 201). The light sources 212a,b may be located in recesses along the sleeve 206, and/or may be flush with the outer surface of the sleeve 206. The light sources 212a,b may LEDs. Optionally, filters may be disposed over the side imaging element and/or the light sources, as described for other embodiments herein.

The secondary imaging endoscopic device 204 may have a distal lip 208 that is configured to engage with the distal end of the endoscope 200. Optionally, a pressure and/or force sensor may be located on the distal lip 208 so that the contact force between the distal-most edge of the secondary imaging endoscopic device 204 and the wall of the body cavity may be measured and conveyed to the controller and/or practitioner. Optionally, the secondary imaging endoscopic device 104 may also comprise an accelerometer 218 located on the sleeve 206. The data from the accelerometer 118 and/or the pressure sensor and/or force sensor may be wirelessly communicated to a controller at a proximal end. Alternatively, the data from these sensors, along with the image data from the side imaging elements may also be communicated to a proximal controller via an electrical conduit 220 enclosed within an elongate tube or catheter 216, as depicted in FIG. 2F. The electrical conduit 220 may also convey signals from the controller that operate the pivot mechanism of the side imaging elements and/or light source, adjust the intensity of the light sources, activate or deactivate the side imaging elements and/or light sources, etc. In this variation, the electrical conduits of the first and second side imaging elements and light sources may be enclosed within a single control cable or tube 216, instead of two separate control cables or tubes depicted in the previous embodiment. The electrical conduits 220a,b may be, for example, one or more wires and/or a flexible circuit board, that extend along the length of the tube 216. In some variations, the electrical conduits 220a,b may share a common substrate, for example, a layered flexible PCB. The tube 216 may optionally comprise additional longitudinal channels or lumens for surgical tools and/or fluid infusion, as described above. The electrical conduit and/or control cable of a secondary endoscopic imaging device may be separate and/or independent from the electrical conduits and/or control cables of the main endoscope. Although the control cable or tube depicted here extends longitudinally along an external surface of the endoscope, in other variations, the control cable or tube may be located within a lumen of the main endoscope and/or be embedded within the main endoscope.

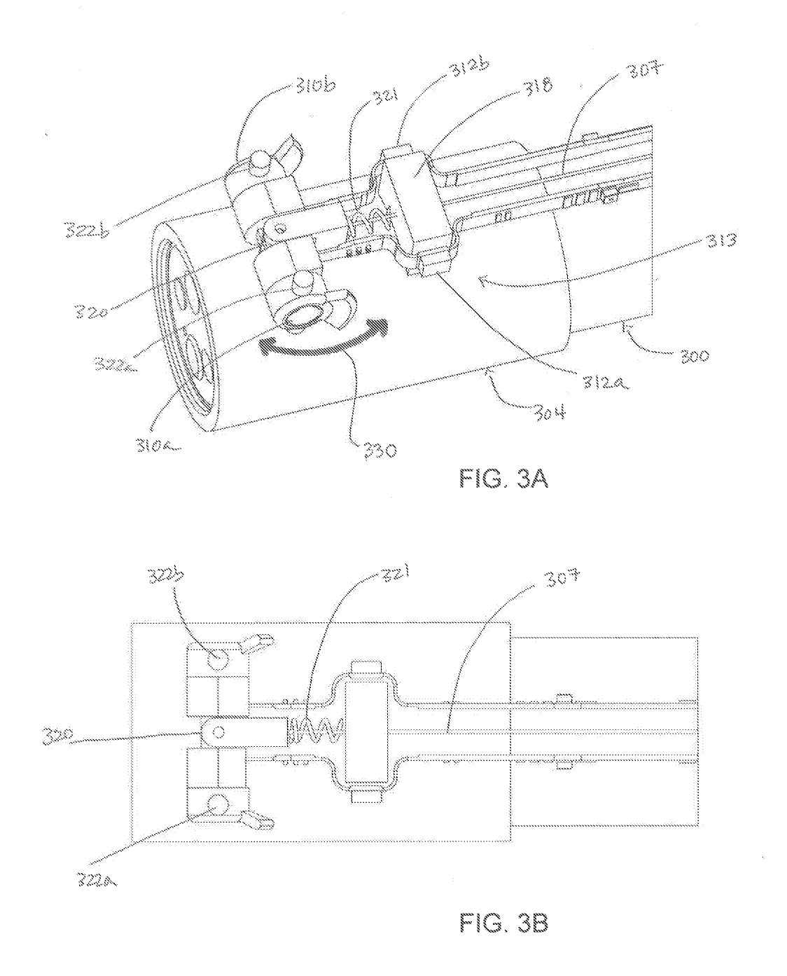

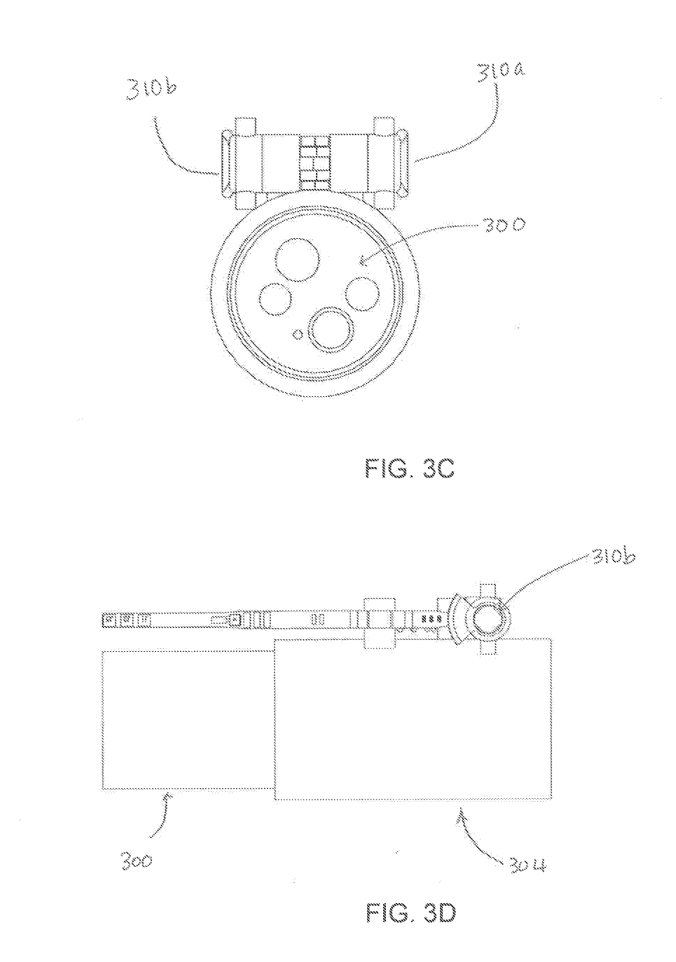

FIGS. 3A-3D depict another variation of a secondary imaging endoscopic device 304 disposed over an endoscope 300, where the side imaging elements of the secondary imaging endoscopic device are rotatable about an intrinsic axis (i.e., a pivot axis) or a extrinsic axis (i.e., an orbital axis) to adjust their field of view. The movement mechanism depicted in FIGS. 3A-3D and described below may be adapted for use in any of the secondary imaging endoscopic devices described herein. The secondary imaging endoscopic device 304 may comprise a sleeve 306, a first side imaging element 310a, a first light source 312a, a second side imaging element 310b and a second light source 312b, and a displacement mechanism 313. Optionally, the secondary imaging endoscopic device 304 may also comprise an accelerometer 318, as described previously. The location of the first and second side imaging elements and first and second light sources may be similar to that described and depicted in FIGS. 2A-2F. The displacement mechanism 313 and underlying components of the first and second side imaging elements and light sources may be enclosed in a liquid-tight housing (not shown), where the housing may have recesses and grooves similar to the recesses in the sleeves of the variations described above. The first and second side imaging elements and/or light sources may be rotated in a lateral direction relative to the longitudinal axis of the endoscope (e.g., in proximal-distal direction as indicated by arrows 330), and/or may have additional degrees of freedom (e.g., rotated 360 degrees). The displacement mechanism 313 may comprise an actuation pivot 320, a shape memory actuator wire 307 connected to the actuation pivot, a return spring 321 disposed over the shape memory actuator wire, a first imaging element pivot hinge 322a attached to the first side imaging element 310a and a second imaging element pivot hinge 322b attached to the second side imaging element 310b. The contraction and expansion of the shape memory actuator wire may be controlled by adjusting an electric potential applied to the two ends of the wire. For example, the shape memory actuator wire may be a muscle wire (such as Muscle Wire® by Dynaolloy of Tustin, Calif.). The actuation pivot 320 may be attached to the first and second imaging element pivot hinges 322a,b such rotating the actuation post or pin 320 causes each of the imaging element pivot hinges 322a, 322b to rotate together and adjust the field of view of the first and second side imaging elements. In other variations, the imaging element pivot hinges may be attached to separate actuation posts or pins that are independently controlled, thereby allowing the side imaging elements to be pivoted independently. The muscle wire 307 may expand or contract longitudinally as controlled by a potential applied by a proximal controller, where lateral expansion and contraction of the muscle wire 307 is translated to an angular rotation of the actuation pivot 320. The return spring 321 acts to bias the actuation pivot 320 to a default position in the absence of an expansion and/or contraction force on the muscle wire 307. Other suitable displacement mechanisms may also be used to translate, rotate and/or pivot the side imaging elements, including pivot mechanisms that comprise ball bearings, etc. The displacement mechanism described above may also be used to move the light sources in conjunction with (or alternatively, independently from) the side imaging elements.

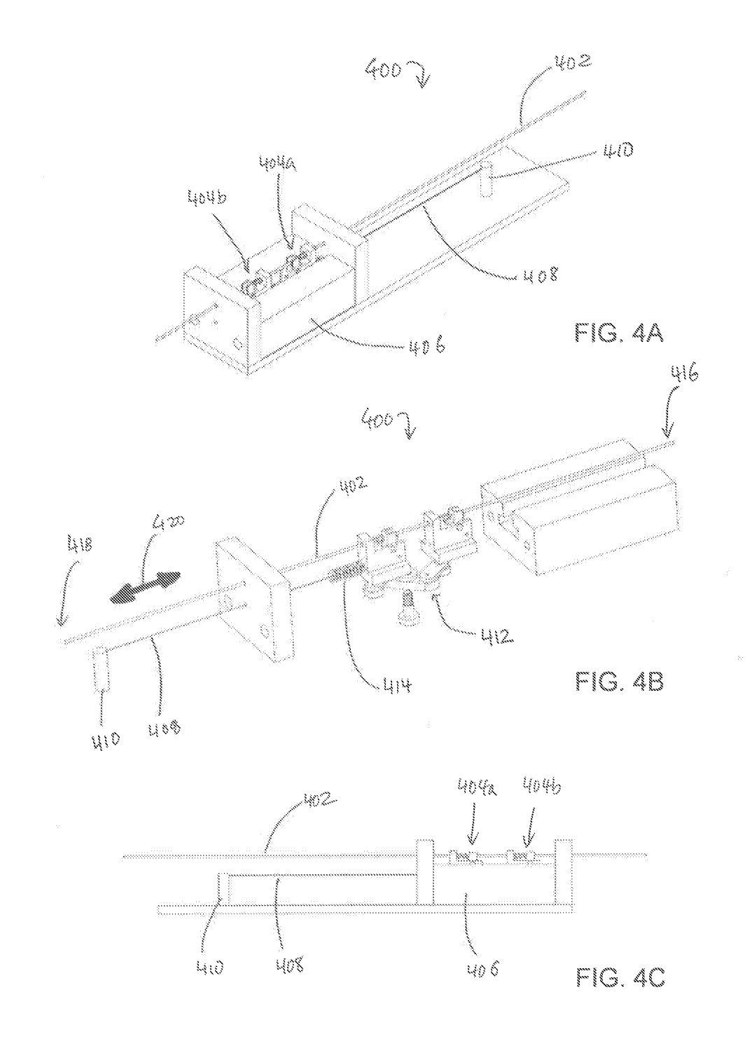

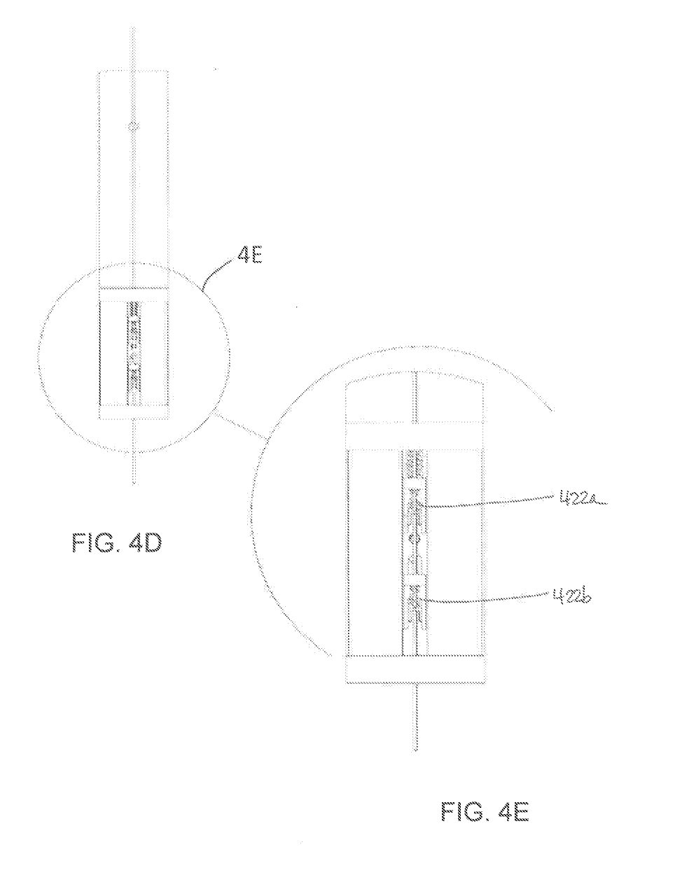

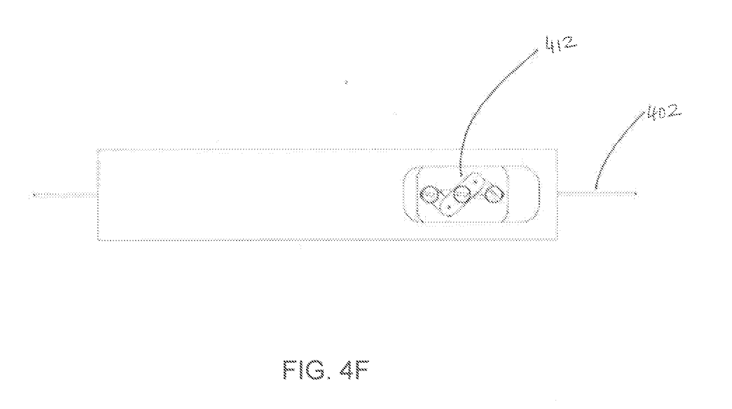

Another mechanism that may be included with a secondary imaging endoscopic device for adjusting the position of the side imaging elements of the secondary imaging endoscopic device is depicted in FIGS. 4A-4F. The pivot mechanism 400 may comprise an actuation wire 402, a first actuation wire carriage 404a, a second actuation wire carriage 404b, a carriage base 406 that retains the first and second actuation wire carriages, and a muscle wire 408 attached to the first actuation wire carriage 404a on one end and attached to a post 410 on the other end. The mechanism may further comprise a hinged cross-linker 412 that couples the first and second carriages together such that lateral motion of one carriage causes a corresponding lateral motion of the other carriage, and a return spring 414 that biases the position of the carriages to a desired location. The mechanism 400 may be enclosed in a housing and attached to a sleeve of a secondary imaging endoscopic device, similar to the sleeves and housing described and depicted above. The actuation wire 402 may be threaded through openings in the first and second carriages and releasably retained in those openings, e.g., by pinching, and extended in the carriage by extension springs 422a, b. The proximal portion 416 of the actuation wire 402 may be attached to a proximal post (not shown), while the distal portion 418 of the actuation wire 402 may be attached to an activation pivot or hinged imaging element pivot, as described above. Electrical activation of the muscle wire 408 by a proximal controller (either in a wireless or wired configuration) may cause expansion and contraction of the muscle wire such that the first and second carriages are laterally translated. Repeated and/or periodic electrical activation of the muscle wire 408 to cause repeated and/or period expansion and contraction of the muscle wire may act to move the first and second carriages such that the actuation wire 402 is translated in a lateral direction. For example, the actuation wire may be laterally advanced forward and backward in the directions indicated by arrow 420, which may in turn cause a side imaging element pivot to rotate and change the field of view of the side imaging element. A stop (not shown) may be provided on the actuation wire 402 to limit the degree to which the actuation wire is translated in a forward or backward direction. While forward and backward motion of the actuation wire may be attained using the mechanism described and depicted here, other mechanisms may alternatively be used. For example, there may be two muscle wires that are controlled such that expansion and contraction of a first muscle wire causes the actuation wire to move in a first direction (e.g., forward) and the expansion and contraction of a second muscle wire causes the actuation wire to move in a second direction (e.g., backward). A proximal controller may adjust the electrical activation of the muscle wire 408 according to the images that are acquired such that the overlap between the various imaging elements (e.g., between the side imaging elements of the secondary imaging endoscopic device or between each side imaging element and the front-facing endoscope imaging element) may be kept at a desired value (e.g., up to 1%, 5%, 10%, 15%, 20%, 30%, 45%, etc.).

The mechanism 400 may also be used to extend and/or retract a device that is attached to the distal end of an endoscope. For example, the distal portion 418 of the actuation wire 402 may be attached to a snare, such that moving the actuation wire 402 forward acts to extend the snare (e.g., to encircle a polyp) and retract the snare (e.g., to capture the polyp). Alternatively or additionally, the mechanism 400 may also be used to extend and retract an imaging element of a wireless secondary imaging endoscopic device.

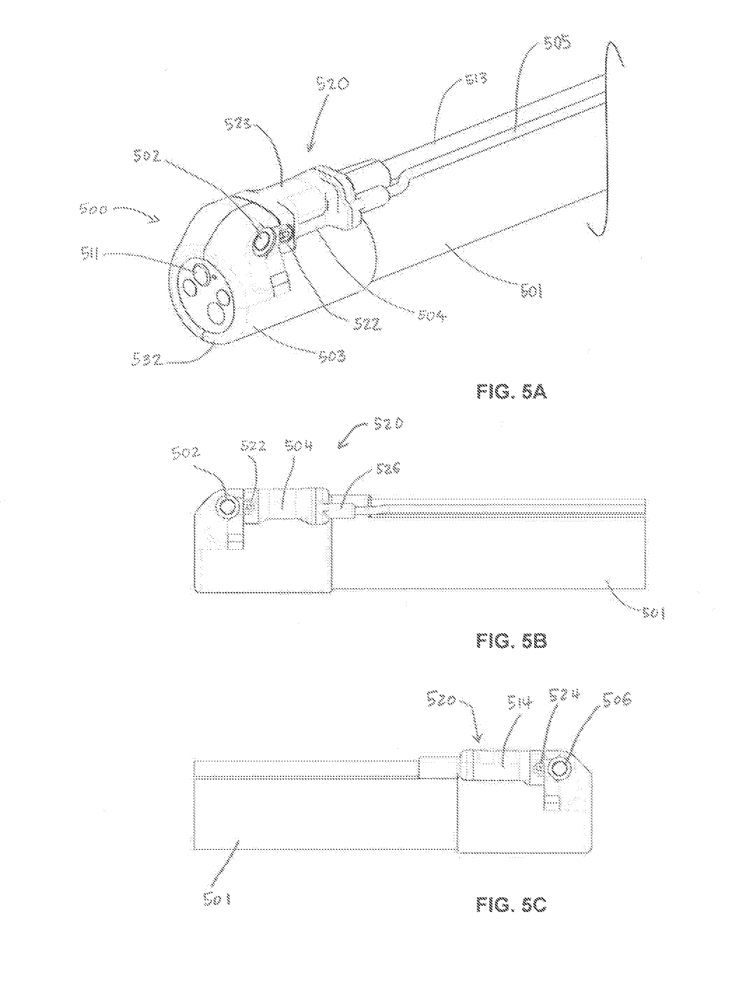

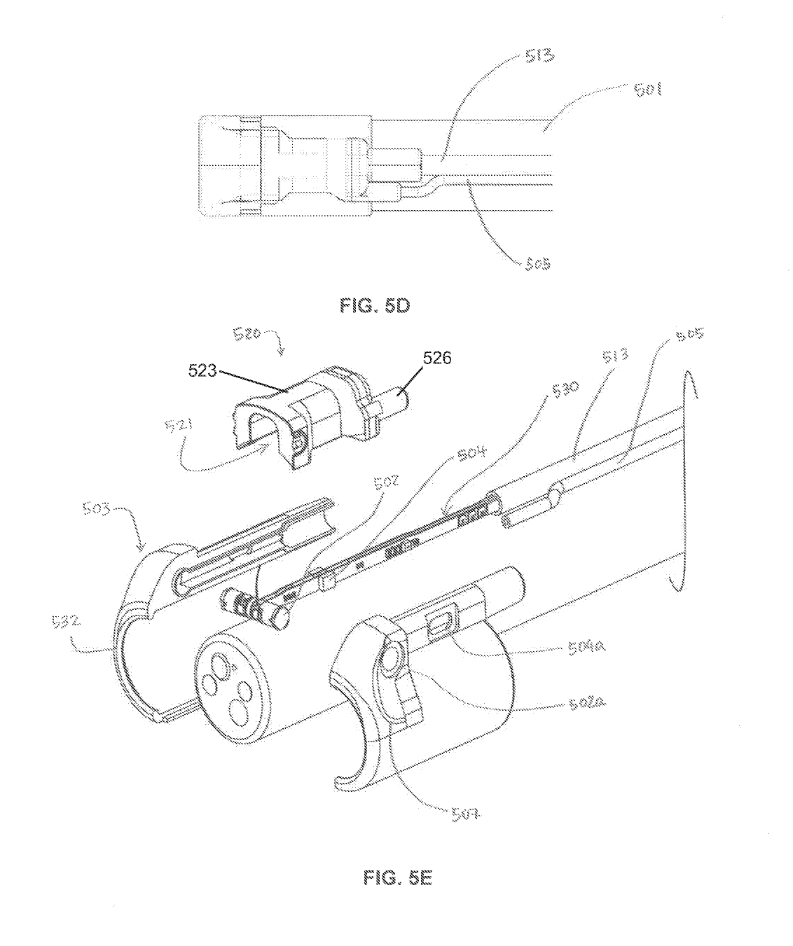

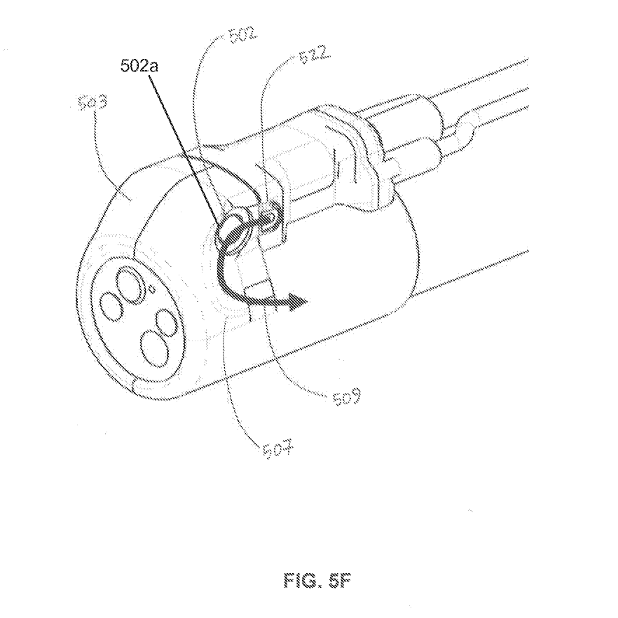

Some variations of a secondary endoscopic device may comprise one or more channels or ports for the passage of fluids therethrough. For example, one or more fluid delivery ports may be provided in the proximity of the side imaging elements so that a solution (e.g., saline) or air may be delivered to the imaging elements for clearing away visual obstructions. One variation of a secondary imaging endoscopic device having a fluid delivery module (which may or may not be detachable is depicted in FIGS. 5A-5E. As depicted there, the secondary imaging endoscopic device 200 may comprise an imaging module having a first side-facing imaging element 502, a second side-facing imaging element 506, a first light source 504 for illuminating the visual field of the first side-facing imaging element, a second light source 514 for illuminating the visual field of the second side-facing imaging element, and a fluid delivery module 520 having a first fluid port 522 adjacent to the first side-facing imaging element and a second fluid port 524 adjacent to the second side-facing imaging element. The side-facing imaging elements for any of the secondary imaging endoscopic devices described herein may comprise a lens assembly and an image detector or sensor (e.g., CMOS or CCD sensor). The lens assembly may optionally comprise one or more filters, and some variations, may include a prism, dichroic mirror, or any suitable optical component. The lens assembly may help to focus images for acquisition by the image detector or sensor. The lens assembly may have a fixed focal depth between about 2 mm to about 45 mm. While the side-facing imaging elements 502, 506 are depicted as having a particular optical configuration, the side-facing elements may have any of the optical configurations depicted and described in FIGS. 17A-17C. The secondary imaging endoscopic device may comprise an imaging module housing 503 that encloses and/or supports the imaging elements 502, 506 and light sources 504, 514, and may comprise an endoscope attachment member such as a sleeve that is sized and shaped to fit over the distal portion of the endoscope 501. For example, the housing may have one or more openings, recesses and curves which may retain the imaging elements and light sources, and may have a lumen therethrough for retaining a distal segment of the endoscope 501. The housing 523 of the fluid delivery module 520 may comprise a lumen 521 therethrough which may be sized and shaped to fit with a corresponding portion of the housing 503. For example, as depicted in FIG. 5E, the housing 523 of the fluid delivery module 520 may have a lumen 521 that has a U-shaped cross-section. The fluid delivery module 520 may also comprise one or more fluid inlet ports 526. The fluid inlet port 526 may be connected to a first tube 505, which may attach to and extend longitudinally along the length of the endoscope 501 may be connected at its proximal end to a fluid reservoir. Although the first tube 505 (e.g., the fluid conduit) is depicted as extending along an outer surface and along the length of the endoscope, in other variations, the tube 505 may be located within a lumen of the main endoscope. The fluid from the reservoir may be transported through the tube and to the inlet port of the fluid delivery module by any suitable means (e.g., the application of positive pressure, pumping, etc.). The fluid delivery module may operate separately and/or independently from any fluid ports of the main endoscope. For example, a practitioner may provide fluid to a fluid delivery module of a secondary endoscopic imaging device without providing fluid to a flush port in the main endoscope (and vice versa). The proximal fluid reservoir may be common between the main endoscope and the secondary endoscopic imaging device, or each may have a separate fluid reservoir.

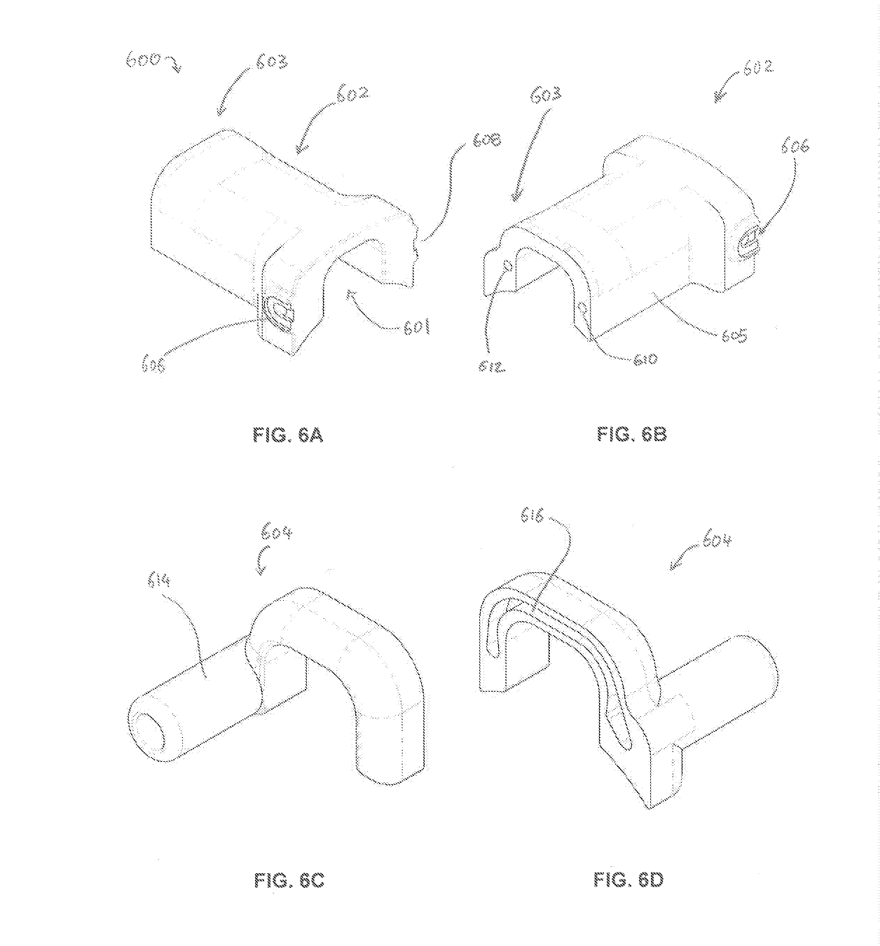

FIGS. 6A-6D depict perspective and component views of one variation of a fluid delivery module of any of the secondary imaging endoscopic devices described herein. The fluid delivery module 600 may have a two-part housing comprising a base portion 602 and an endplate portion 604 (FIGS. 6C-6D). The base portion 602 may have a U-shaped cross-section and comprise one or more fluid outlet ports 606, 608 on either side of a lumen 601. The lumen 601 may be sized and shaped to correspond and to fit with the portion of the secondary imaging endoscopic device housing. As illustrated in FIG. 6D, the endplate 604 may comprise a conduit, channel or cavity 616 (which may be a U-shaped channel or cavity that corresponds with the U-shaped cross-section of the base portion) that is in fluid connection with the inlet port 614. The fluid outlet ports 606, 608 may be connected to lumens and/or channels within the walls of the base portion, where the lumens and/or channels terminate at openings 610, 612 on the proximal side 603 of the base portion 602. When the endplate 604 is attached to the proximal side 603 of the base portion 602 (e.g., by any fluid-tight mechanism such as using adhesives, welding, soldering, and the like), the openings 610, 612 may be aligned and/or in fluid communication with the cavity 616. Fluid injected from a proximal reservoir (not shown) may be transported through a tube along the length of the endoscope, through the inlet port 614, and into the cavity 616, which then distributes the fluid through the cavity 616 to the openings 610, 612 to both of the outlet ports 606, 608. The cavity 616 may serve to divide a single fluid path into two fluid paths, so that a single fluid inlet can supply fluid to two outlet ports. Optionally, some variations of a fluid delivery module housing may comprise one or more inflatable membranes (e.g., balloons) attached to either or both the base portion 602 and/or endplate 605. For example, balloons may be located on either or both sides of the fluid delivery module, proximal to the outlet ports 606, but distal to the inlet port 614. The balloons may be inflated with a fluid (e.g., gas or liquid) as may be desirable to ensure a space between the surface of the secondary imaging endoscopic device and the wall of the body lumen under examination. The inflation fluid may be provided via the infusion tube (e.g., first tube 505), in the same lumen or a different lumen used to delivery fluid to the outlet ports.

The housing of a fluid delivery module may be made of opaque and/or translucent (e.g., transparent) materials. The optical characteristics of certain portions of the module housing may be determined at least in part by the proximity of that portion to an optical element of the secondary imaging endoscopic device. For example, portions of the fluid delivery module that do not overlap with the visual field of a side imaging element or the illumination field of a light source may be made of an opaque material, while portions that cover (at least partly or wholly) a light source and/or imaging element may be translucent (e.g., transparent). For example, the sides 605 of the base portion 602 (which may cover the light source when the secondary imaging endoscopic device is fully assembled) may be made of a transparent material while the endplate portion and other portions of the base may be made of opaque materials. Alternatively, the entire housing of a fluid delivery module may be made of a translucent material. Certain portions of the fluid delivery module may be made of opaque materials to help reduce light scatter and noise, as may be desirable. In some variations, the portion of the housing that covers over the light source of a secondary imaging endoscopic device (e.g., sides 605) may be made of a material that may be configured to diffuse light from the light source. For example, the sides 605 may be a filter, and/or a Fresnel lens, and/or may have an etched/frosted/machined pattern across its surface to diffuse or de-focus light. Such a feature may help to expand the illumination field of the light source. Alternatively or additionally, the light source itself may have a filter and/or optical component that may facilitate the expansion of the illumination field.

Although the fluid delivery module 600 described above has two outlet ports on either side of a U-shaped lumen, it should be understood that the shape of the fluid delivery module housing and the location and number of fluid outlet ports may vary depending on the location and number of imaging elements on a secondary imaging endoscopic device. For example, a secondary imaging endoscopic device may have two side imaging elements that are located at different circumferential locations (e.g., circumferentially across from each other, 180 degrees away from each other, such as is depicted in FIGS. 1A-F). The channel or cavity within the endplate portion may be shaped differently in order to accommodate the different locations of the outlet ports. A secondary imaging endoscopic device having side imaging elements that are located adjacently about the same circumferential location but facing in opposite directions (e.g., such as is depicted in FIGS. 2A-2F, FIGS. 5A-5E) may have a fluid delivery module similar to that described above. Any of the secondary imaging endoscopic devices described herein may optionally comprise a fluid delivery module as described above. Alternatively, although shown as a separate module, structures of any of the fluid delivery modules described herein may be integral with any structures (e.g., housing, imaging module) of the secondary imaging endoscopic device.

Regardless of the number of the side imaging elements, in some variations, the curvature of the housing of the secondary imaging endoscopic device around the side imaging elements may help to guide the fluid flowing from the fluid delivery module such that after the fluid has passed over the imaging element, it flows away from the imaging element. This may help to reduce fluid turbulence (e.g., fluid scatter and/or splash) that may result in image distortions and/or artifacts, and/or may also facilitate sweeping away any debris that adheres to and/or blocks the view of the imaging element. For example, there may be a concave curve in the housing of the secondary imaging endoscopic device around the side imaging element that is angled such that fluid may be directed away from the imaging element after it moves across its surface. FIG. 5E depicts an exploded view of the secondary imaging endoscopic device, where the housing 503 comprises openings 502a, 506a (not shown) for the side image elements 502, 506, openings 504a, 514a (not shown) for the light sources 504a, 514a, and concave curves surrounding each of the openings 502a, 506a. FIG. 5F depicts an enlarged view of the secondary endoscopic device (with the optional fluid module) assembled over an endoscope. The opening 502a for the first side imaging element may be located at the top of the concave curve 507 such that fluid moving across the first imaging element may flow downward along the concave curve (e.g., along the direction indicated by arrow 509). Debris swept off the imaging element may also follow in this fluid path, thereby clearing the field of view of the imaging element.

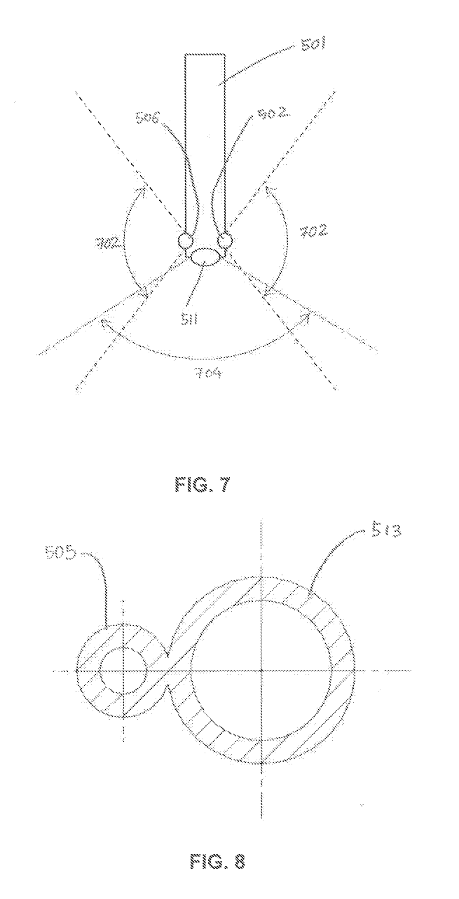

The location of the side imaging elements 502, 506 may be similar to the location of the imaging elements 310a, 310b of the secondary imaging endoscopic device depicted in FIGS. 2A-2C. The side imaging elements may be pivotable (e.g., such as imaging elements 310a,310b) or fixed (e.g., the side imaging elements 502, 506). The views of the first and second side imaging elements 502, 506 of the secondary imaging endoscopic device 500 may be similar to the views of the first and second side imaging elements 310a, 310b. FIG. 7 is a schematic depiction of the field of views of the first and second side-facing imaging elements, and the field of view of the primary endoscope (i.e., front-facing) imaging element 511. The viewing angle 702 of each of the side imaging elements may be from about 120 degrees to about 150 degrees, e.g., about 130 degrees or 135 degrees. As depicted there, a side-facing imaging element may provide both side views and rear/retrograde views of the area around the endoscope. The viewing angle 704 of the primary endoscope (front-facing) imaging element 511 may be from about 125 degrees to about 155 degrees, e.g., about 140 degrees. The field of view of the side imaging elements and the endoscope main imaging element may overlap, where the angle over overlap may be from about 2 degrees to about 30 degrees, e.g., about 5 degrees, about 10 degrees. The first and second side imaging elements 502, 506 may be located about 2 mm to about 5 mm from the distal-most end of the endoscope 501, e.g., about 5 mm. Images acquired by first and second side imaging elements that are relatively close to the distal end of the endoscope may be more intuitively interpreted by a practitioner who is simultaneously viewing videos taken from the main front-facing imaging element and the side imaging elements. Providing a degree of overlap between the images/videos acquired by the secondary side and main front imaging elements may also provide images that are readily understood by a practitioner.

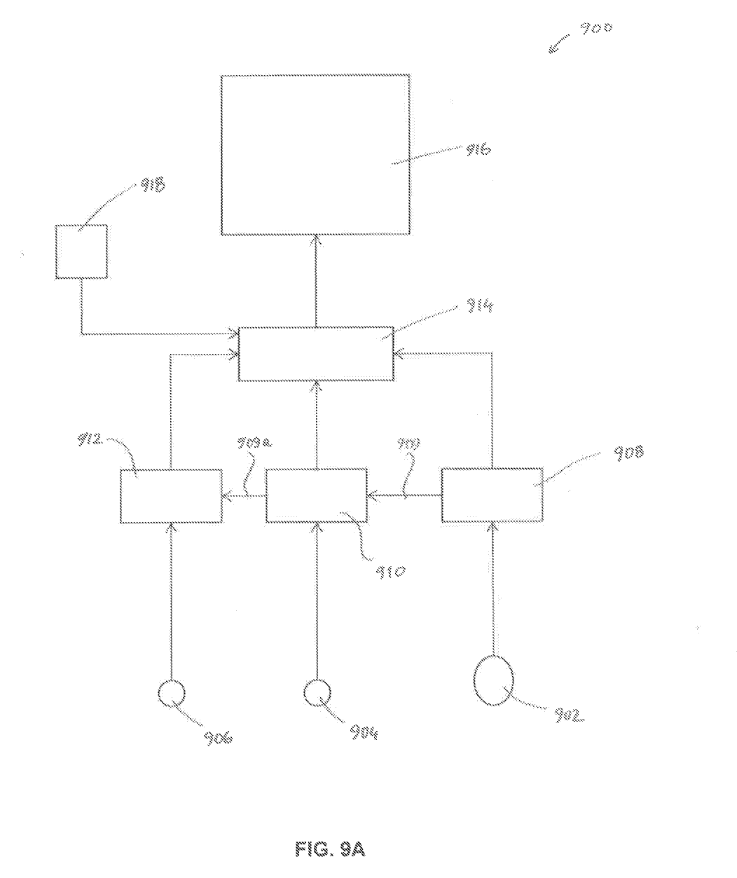

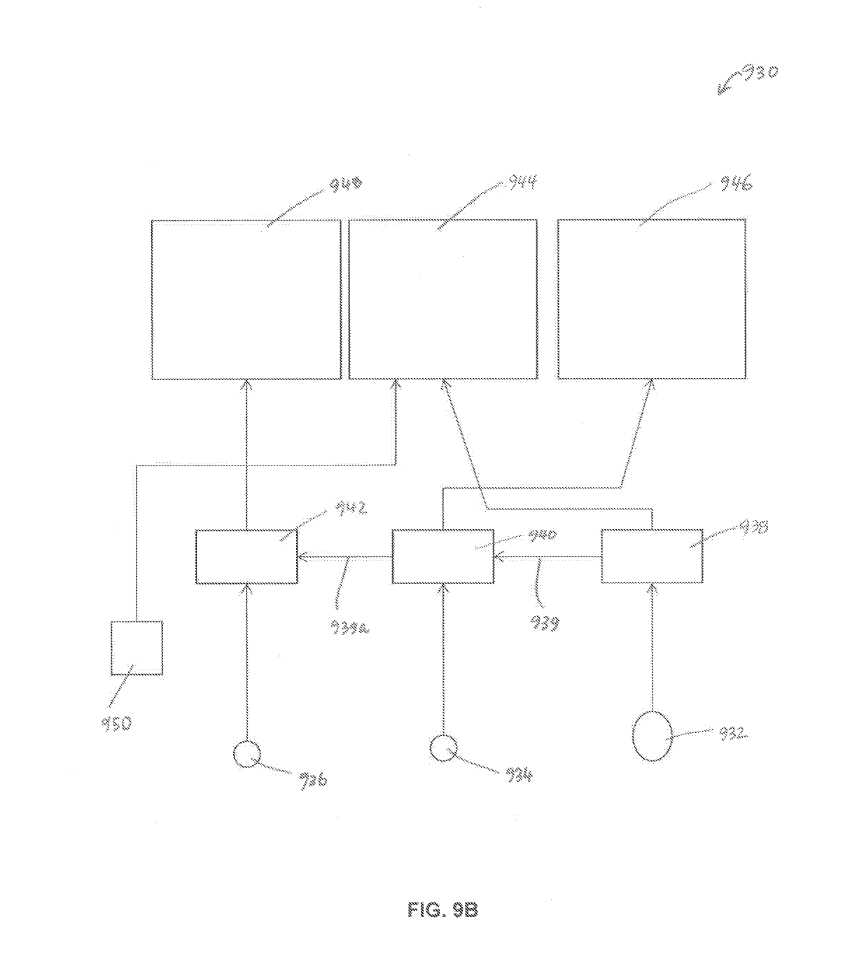

As described above, a first tube 505 attached to the endoscope 501 may be provided from a proximal fluid reservoir along the length of the endoscope to supply fluid to the fluid delivery module 520. Optionally, a second fluid-tight tube 513 (e.g., a control cable) may be attached to and provided along the length of the endoscope 501 to provide a conduit for the electrical connections between the side imaging devices, light sources and a proximal controller. For example, the image data from the side imaging elements may be communicated to a proximal controller via an electrical conduit 530 enclosed within the control cable or elongate tube 513, as depicted in FIG. 5E. The tubes 505, 513 (as well as any of the tubing described herein) may be made of any suitable materials, including polymeric materials such as PEBAX (55D). The electrical conduit 530 may also convey signals from the controller that adjust the intensity of the light sources, activate or deactivate the side imaging elements and/or light sources, etc., and may be similar to the control cables and/or electrical conduits 220a,b as previously described. Although the electrical conduit 530 (e.g., flexible PCB) is depicted as extending longitudinally proximally from the housing of the secondary imaging endoscopic device (e.g., may extend proximally to a proximal connector or controller port), in other variations, the electrical conduit may be entirely contained within the length of the housing of the secondary imaging endoscopic device, or may be generally the same length as the housing. In still other variations, an electrical conduit may extend part of the way between the housing and the proximal controller or port. Optionally, an electrical conduit may comprise a longitudinally extending electrical wire or control cable that may connect the flexible PCB to the proximal controller of the secondary imaging endoscopic device. The electrical conduits (e.g., either or both the flexible PCB or the wire) of the imaging elements and light sources may be enclosed within a single lumen of a single tube, or may be located within two separate tubes, or in different lumens of the same tube. In some variations, the first tube 505 and the second tube 513 may be connected together (as depicted FIG. 8) or may be separate. For example, having a fluid perfusion tube that is separate from the tube housing electrical conduits may allow the fluid perfusion tube to be removed and disposed of (e.g., after use in a patient), while the electrical conduit tube may be retained and sterilized for additional uses. Optionally, either or both of the first and/or second tubes may comprise additional lumens for the delivery of multiple types of fluids (e.g., cleaning fluids, image contrast agents, gaseous fluids, etc.) and/or surgical tools and/or electrical components. In still other variations, the secondary imaging endoscopic device may comprise a wireless transmitter, and imaging elements and light sources may be wirelessly powered and/or controlled. In such variations, it may not be necessary to have a tube for housing electrical wires, since power and control of the electrical components of the secondary imaging endoscopic device are provided via wireless transmissions. The tubes 505, 513 may be slidably coupled to the elongate body of the endoscope via clips (e.g., C-clips). The clips may be secured to the endoscope (e.g., using tape or other adhesive mechanisms) so that the clips cannot move longitudinally along the length of the endoscope. The tubes 505, 513 may be coupled to the clips such that the tubes can slide with respect to the endoscope (e.g., to accommodate the bends and curves of the endoscope as it is advanced within a body lumen). The electrical conduits and/or control cables of a secondary endoscopic imaging device may be separate and/or independent from the electrical conduits and/or control cables of the main endoscope. Although the control cables or tubes depicted here extend longitudinally along an external surface of the endoscope, in other variations, the control cables or tubes may be located within a lumen of the main endoscope and/or be embedded within the main endoscope.

The secondary imaging endoscopic device may be attached to the main endoscope in various ways such that the orientation of the secondary imaging elements with respect to the main imaging element of the endoscope is fixed. The secondary imaging endoscopic device may be snap-fit, friction-fit, screw-fit, compression-fit, and/or clamped and/or otherwise releasably secured to the endoscope. In some variations, the secondary imaging endoscopic device may be attached such that the position of the secondary imaging elements is still adjustable (e.g., rotatable about the longitudinal axis of the endoscope), and then subsequently locked once the desired location and/or orientation with respect to the main imaging element has been attained. For example, the housing 503 of the secondary imaging endoscopic device may comprise a distal lip 532 that circumscribes the distal end of the endoscope such that the distal edge of the endoscope snaps into a ridge or recess in the distal lip 532. The sleeve portion of the housing 503 may be disposed over the distal portion of the endoscope and engaged to the endoscope by snapping the distal lip over the distal end of the endoscope. The fluid delivery module may be attached to the housing 503 before or after the housing 503 is coupled to the endoscope. Alternatively or additionally, the housing 503 may comprise two parts 503a and 503b (that may be bilaterally symmetric) that snap together around the distal portion of the endoscope 501 such that when the two parts are engaged and fitted over the endoscope. After the secondary imaging endoscopic device (with the optional fluid delivery module) is attached to the endoscope, it may still be rotatable about the longitudinal axis of the endoscope. The practitioner may then rotate the secondary imaging endoscopic device (with the optional fluid delivery module) until the desired viewing orientation of the side imaging elements is attained. The internal surface of the secondary imaging endoscopic device housing may comprise a material with a relatively high coefficient of friction such that the secondary imaging endoscopic device can be rotated only using rotational forces much greater than those that might be encountered during use in a body lumen. For example, the internal surface of the housing may comprise a tacky material grips the outer surface of the endoscope. Alternatively or additionally, a removable adhesive may be used to attach the secondary imaging endoscopic device to the endoscope. For example, before the adhesive sets, the position of the secondary imaging endoscopic device relative to the main endoscope imaging element may be adjusted (e.g., rotated), but after the adhesive sets, the position of the secondary imaging endoscopic device may no longer be adjusted. The adhesive may provide a secure attachment for one or more uses, after which it may be replaced or refreshed with additional adhesive. In some variations, the housing 503 may be fitted over the distal end of the endoscope as described above, and the fluid delivery module may be used to fix the desired orientation. For example, attaching the fluid delivery module may act to compress the sleeve portion of the housing more closely together (or, in the embodiment with two parts 503a and 503b, draw the two parts closer together), which may tightly engage (e.g., by friction and/or compression fit) the endoscope such that the secondary imaging endoscopic device is no longer rotatable. Once the desired orientation has been secured, the tubes for any electrical conduits/wiring or control cables, as well as fluid delivery conduit(s) or lumen(s) may be attached to the endoscope (e.g., using clips, as described above) and connected to proximal controllers and/or fluid reservoirs.

While the sleeve portion of a secondary imaging endoscopic device may form a closed loop such that it fully circumscribes the endoscope (such as the sleeve portion of housing 503 described and depicted above in FIGS. 5A-5D), in other variations, the sleeve portion may not fully circumscribe the endoscope. For example, the sleeve portion may not form a closed loop, but may instead form an open loop or C-shape that partially circumscribes the endo scope, and may secure the secondary imaging endoscopic device to the endoscope via a clamping mechanism. This may allow the secondary imaging endoscopic device to be installed over multiple endoscopes with different circumferences and diameters. The C-shaped sleeve or clip may comprise a living hinge with shape memory that deflects during installation of the secondary imaging endoscopic device over the endoscope, but then returns to its initial position to secure the secondary imaging endoscopic device to the endoscope. The C-shaped sleeve portion may secure the secondary imaging endoscopic device to the endoscope such that the secondary imaging endoscopic device is not rotatable about the endoscope. Alternatively, the secondary imaging endoscopic device may still be rotatable about the endoscope after installation, and rotationally secured using any of the mechanisms described above.

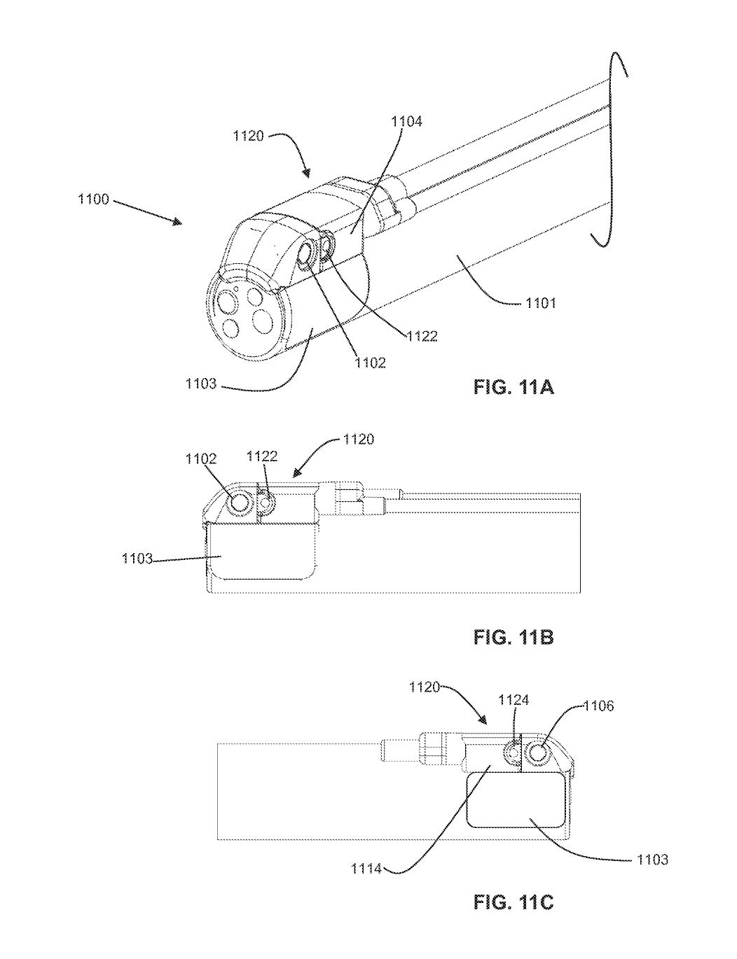

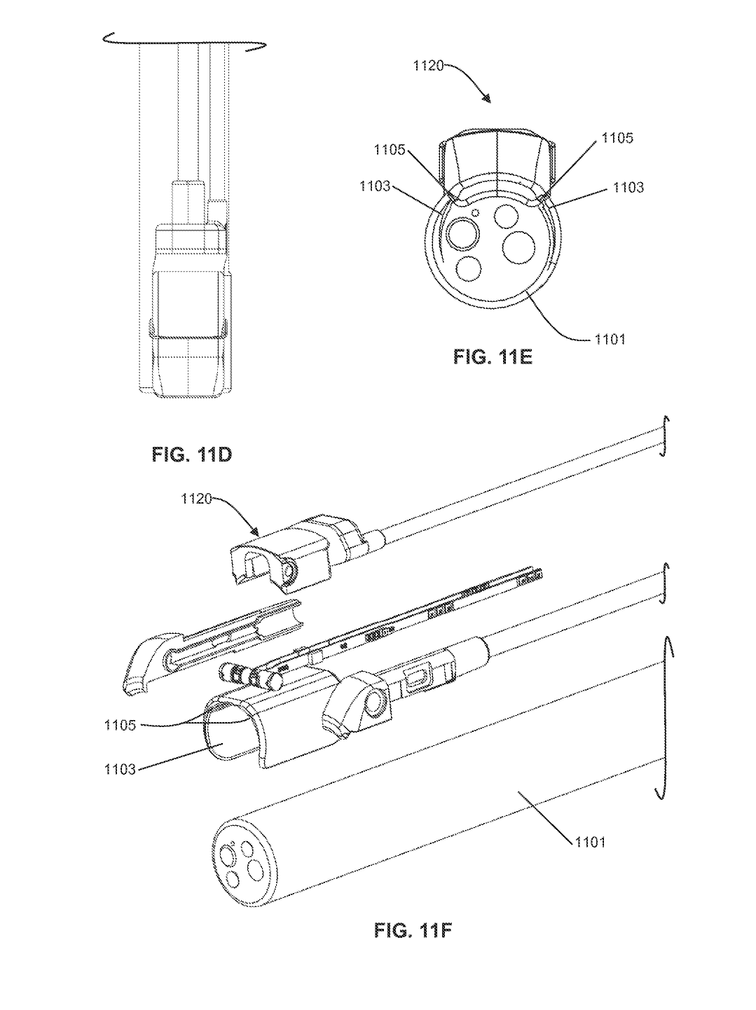

One example of a secondary imaging endoscopic device 1100 comprising a housing having a C-shaped sleeve portion or clip is depicted in FIGS. 11A-11F. The secondary imaging endoscopic device 1100 may comprise a housing having a C-shaped sleeve portion or clip 1103, an imaging module having a first side-facing imaging element 1102, a first light source 1104 adjacent to the first side-facing imaging element, a second side-facing imaging element 1106, a second light source 1114 adjacent to the second side-facing imaging element, and a fluid delivery module 1120. As illustrated in FIGS. 11A-11C, the C-shaped sleeve portion or clip 1103 of the housing does not fully circumscribe the distal portion of the endoscope 1101. The fluid delivery module 1120 may comprise a first fluid port 1122 that is adjacent to the first imaging element 1102 and a second fluid port 1124 that is adjacent to the second imaging element 1114. The side-facing imaging elements 1102 and 1106 of the imaging module may have any of the optical configurations depicted in FIGS. 17A-17C. The fluid delivery module 1120 may be similar to any of the fluid delivery modules described above. FIGS. 11E and 11F depict two longitudinal notches or grooves 1105 along a top portion of the C-shaped sleeve 1103 that may allow the one or both sides of the C-shaped sleeve to deflect outward from their initial state (i.e., such that when deflected outward, the circumference of the C-shaped sleeve increases) during installation. For example, a separating tool may be used to deflect the sides outward. When the outward force deflecting the sides of the C-shaped sleeve has been released, the notches/grooves 1105 may allow the sides of the C-shaped sleeve to revert to their initial state to secure the secondary imaging endoscopic device to the endoscope. Once the C-shaped sleeve has been clamped over the endoscope, the secondary imaging endoscopic device may not be rotatable about the endoscope.

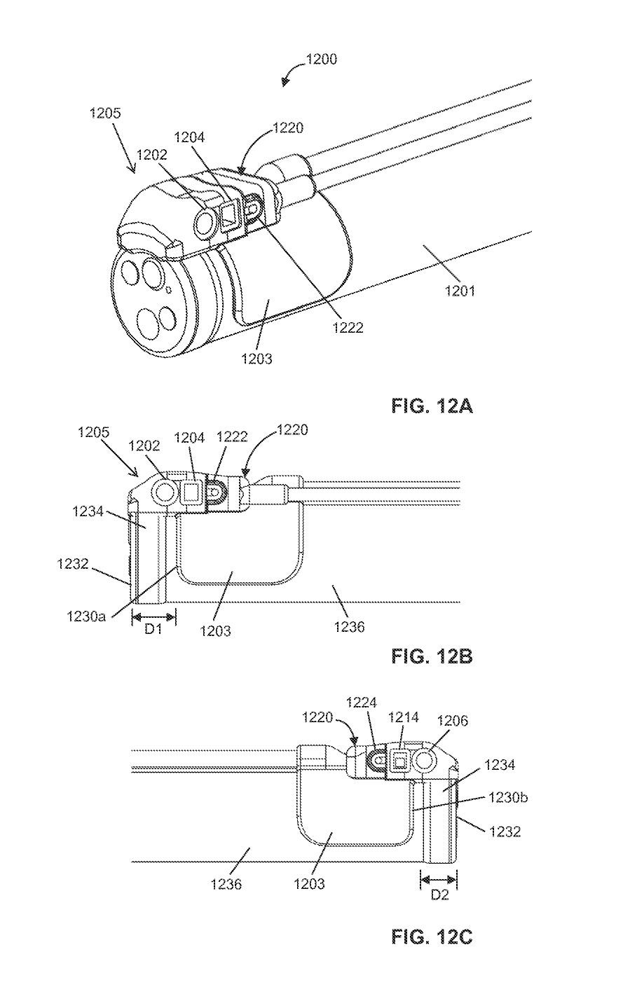

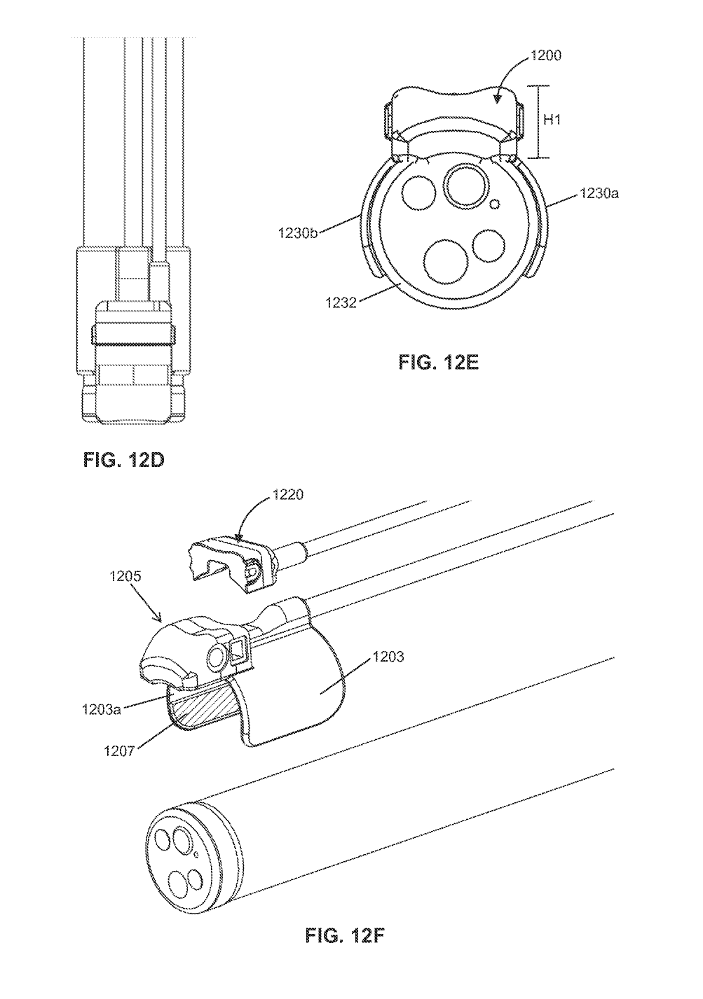

While the secondary imaging endoscopes described above have a fluid port located between the imaging element and the light source, in other variations, the light source may be directly adjacent to the imaging element without a flush port between them. That is, there may be no intervening component (e.g., port, lumen, attachment member, etc.) between the imaging element and light source that may interfere with the amount of light from the light source provided to the field of view of the imaging element. For example, the distance between the imaging element and the light source may be no more than about 3 mm, and may be less than about 2 mm or less than about 1 mm. This may help the field of illumination of the light source coincide more closely with the field of view of the imaging element (e.g., provide a greater area of overlap between the fields of illumination and view). The flush port may be located proximal to both the imaging element and the light source, and may be configured to direct fluid across both the imaging element and the light source. One non-limiting example of a secondary imaging endoscopic device 1200 comprising a light source adjacent to the imaging element is depicted in FIGS. 12A-12F. The secondary imaging endoscopic device 1200 may comprise a housing having a C-shaped sleeve portion 1203, an imaging module 1205 having a first side-facing imaging element 1202, a first light source 1204 directly adjacent to the first side-facing imaging element, a second side-facing imaging element 1206, a second light source 1214 directly adjacent to the second side-facing imaging element, and a fluid delivery module 1220. As illustrated in FIGS. 12A-12C, the C-shaped sleeve portion 1203 of the housing does not fully circumscribe the distal portion of the endoscope 1201. The fluid delivery module 1220 may comprise a first fluid port 1222 that is proximal to the first light source 1204 and a second fluid port 1224 that is proximal to the second light source 1214. The side-facing imaging elements 1202 and 1206 of the imaging module may have any of the optical configurations depicted in FIGS. 17A-17C. The fluid delivery module 1220 may be similar to any of the fluid delivery modules described above. The control cable and/or electrical conduit, as well as the fluid conduit may be similar to the control cables, electrical conduits, and fluid conduits as described above.

The clip or C-shaped sleeve may be configured such that when installed on the insertion tube of the endoscope 1201, the distal-most edges 1230a, 1230b of the clip 1203 is proximal to a rim 1234 of the endoscope (FIGS. 12B-C). In some endoscopes, the rim 1234 may be made of a material that is more slippery than the tubular body 1236 of the endoscope. Engaging the clip proximal to the rim 1234 and/or on the tubular body 1236 of the endoscope may help encourage a more stable engagement with the endoscope. For example, the distance D1 between the distal-most edges 1230a, 1230b of the clip and the distal-most edge of the endoscope 1201 may be from about 2 mm to about 10 mm, e.g., about 4 mm, or about 6.4 mm. In some variations, the imaging elements 1202, 1206 may be located just proximal to or over the rim 1234 of the endoscope when installed. For example, the imaging elements 1202, 1206 may be distal to the distal-most edges 1230a,b of the clip such that the distance D2 between the center of the imaging elements 1202, 1206 to the distal-most edge 1232 of the endoscope is from about 3 mm to about 8 mm, e.g., less than about 5 mm, about 5 mm, about 5.3 mm, about 6.3 mm, about 6.4 mm, etc.

Optionally, the clip or C-shaped sleeve portion 1203 may comprise a region 1207 having a greater coefficient of friction located in the interior surface of the sleeve. A region 1207 of increased friction may help the clip to engage with the insertion tube of the endoscope 1201 such that once engaged, the secondary imaging endoscopic device does not twist around or slide across the endoscope. Although the region 1207 is depicted along a portion of the interior surface 1203a of the clip 1203 in FIG. 12F, it should be understood that the region 1207 can include all or almost all of the interior surface of the clip. For example, a first side of the clip 1203 may have a first region of increased friction and a second side opposite the first side may have a second region of increased friction. The region 1207 may comprise a coating of an adhesive material or glue, such as UV adhesives (e.g., Loctite 3211, Loctite 3321), cyanoacrylates (e.g., 3M CA40, Loctite 4310 Flashcure, Loctite 4311 Flashcure), or 2-part epoxy (e.g., Loctite M-31CL Medical grade epoxy). Alternatively or additionally, the region 1207 may comprise a layer of a tacky material, for example, EPR or EPDM rubbers and/or other tacky or sticky materials. Once the C-shaped sleeve has been clamped over the endoscope, the secondary imaging endoscopic device may not be rotatable about the endoscope. The housing of the secondary imaging endoscopic device may comprise one or more concave curves around the imaging element (e.g., such as the curves described and depicted above in FIG. 5F), or may not comprise any concave curves around the imaging element. The other depicted components of the secondary imaging endoscopic device may be similar to the corresponding components described previously.

The secondary imaging endoscopic device 1200 may have a height H1 from about 1.5 mm to about 5 mm, e.g., 4 mm, 4.2 mm, 4.4 mm, as depicted in FIG. 12E.

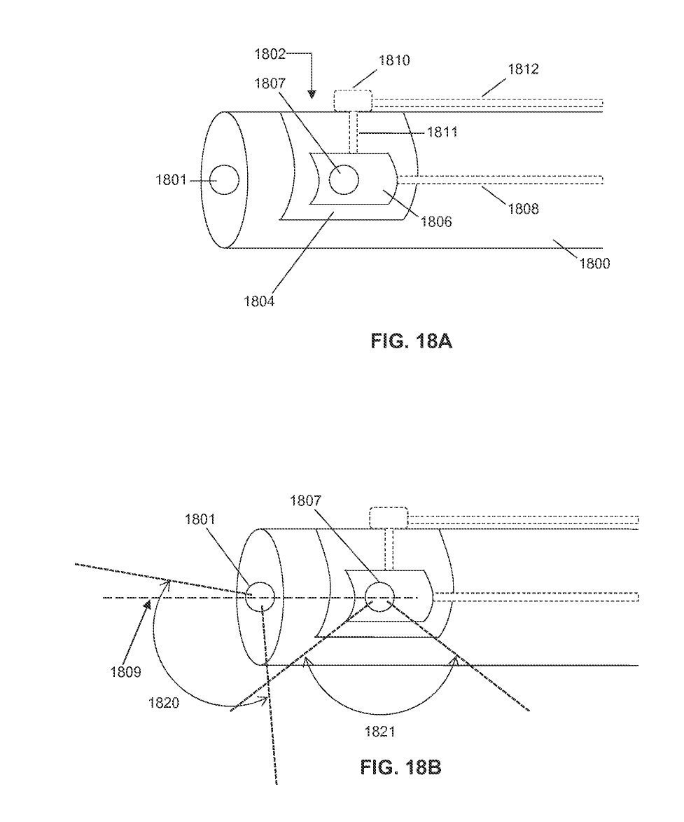

While the secondary endoscopic imaging devices described above have imaging modules such that the side-facing imaging elements are located on a top portion of the main endoscope (e.g., such that the visual axes of the side-facing imaging elements are approximately tangential to the circumference of the main endoscope), in other variations, the side-facing imaging elements may be located such that their visual axes are located on a side portion of the main endoscope (e.g. such that the visual axes of the side-facing imaging elements are approximately perpendicular to the circumference of the main endoscope). Referring back to FIG. 16A, the secondary endoscopic imaging devices described above have side-facing imaging elements that are located higher up on the y-axis than the front-facing imaging element of the main endoscope (i.e., the locations of the side-facing imaging elements are not co-linear with the front-facing imaging element). Alternatively, other variations of secondary endoscopic imaging devices may have side-facing imaging elements that are located at the same level on the y-axis as the front-facing imaging element of the main endoscope (i.e., the locations of the side-facing imaging elements are co-linear with the front-facing imaging element). Such configuration may help facilitate image stitching algorithms executed by a proximal controller, since the front and side images would be taken at approximately the same height or along the same horizontal plane. FIG. 18A schematically depicts a side view of one variation of a secondary endoscopic imaging device 1802 where the imaging module 1806 has side-facing imaging elements that are co-linear with the front-facing imaging element 1801 of the main endoscope 1800 (only one side-facing imaging element 1807 is depicted for clarity; the second side-facing imaging element may be located on the opposite side of the secondary endoscopic imaging device). FIG. 18B schematically depicts the field of view/viewing angle 1820 of the front-facing imaging element 1801 and the field of view/viewing angle 1821 of the side-facing imaging element 1807. The visual axes of each imaging element (e.g., a line bisecting the viewing angle) may be co-planar, and the location of each of the imaging elements may be co-linear along line 1809. The secondary endoscopic imaging device 1802 may optionally comprise any of the fluid delivery modules described above. The imaging module 1804 may have a PCB and/or control cable and/or electrical conduit 1808 for each side-facing imaging element of the imaging module that extends to a proximal controller, or may have a single PCB and/or control cable and/or electrical conduit 1812 for both side-facing imaging elements that extends to a proximal controller. In variations with a single control cable and/or electrical conduit 1812 for multiple side-facing imaging elements, the image sensors of each side-facing imaging element may each have one or more wires 1811 that connect a hub 1810. The electrical signals from all of the side-facing imaging elements may be combined at the hub 1810 and then transmitted to a proximal controller via a single control cable 1812. Alternatively or additionally, signals from the side-facing imaging elements may be wirelessly transmitted to a proximal controller. The electrical and/or fluid conduits and/or control cables of the secondary endoscopic imaging device may be separate and/or independent from the electrical and/or fluid conduits and/or control cables of the main endoscope. The side-facing imaging elements may have any of the optical configurations depicted in FIGS. 17A-17C, however, it may be preferred to have the optical configurations depicted in FIGS. 17B and 17C, as such configurations may have a smaller width than the width W1 of the configuration depicted in FIG. 17A.

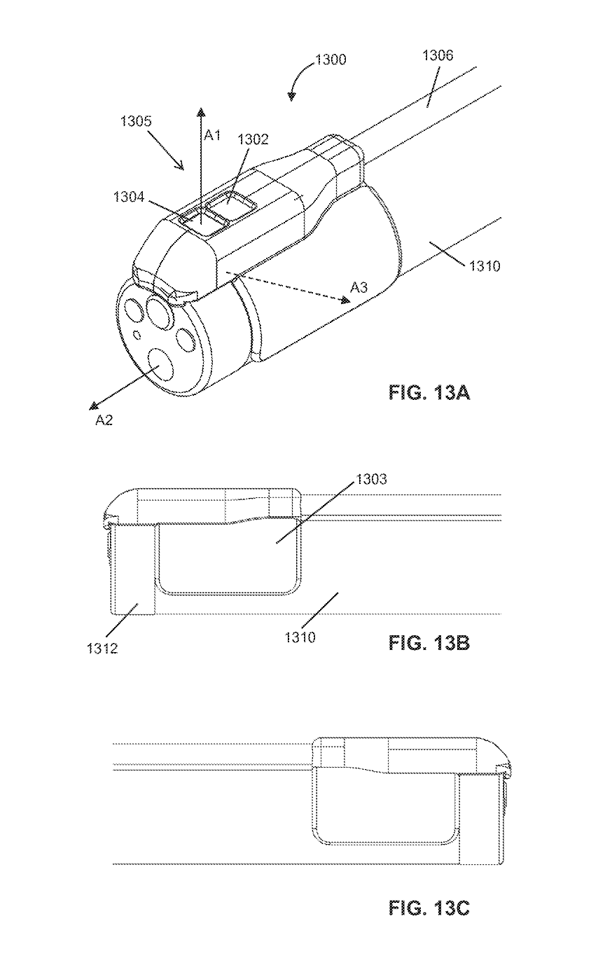

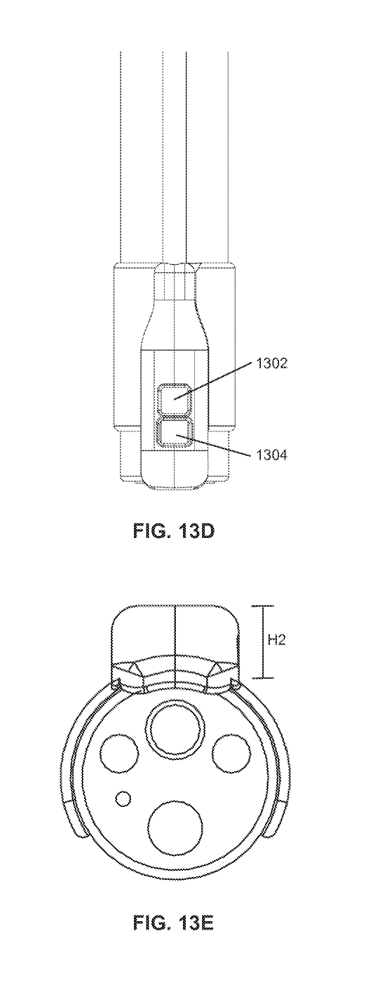

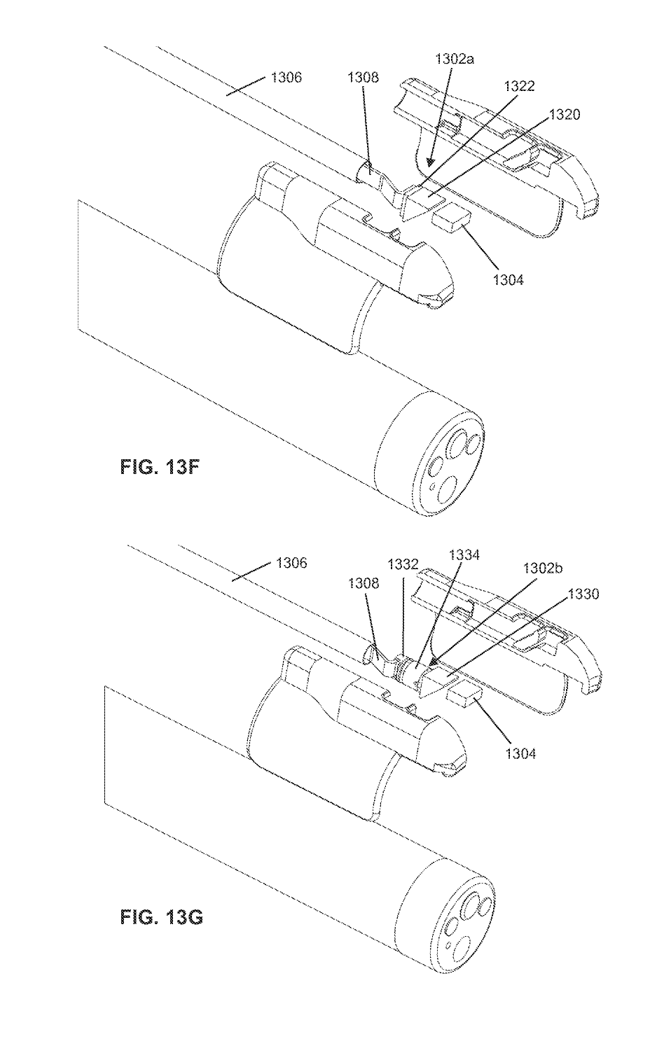

Alternatively or additionally to the side imaging elements and light sources described above, a secondary imaging endoscopic device may comprise an imaging element and a corresponding light source that provides a top field of view (e.g., as depicted and described in FIG. 16A, may have a visual axis that is parallel to, or at an angle less than 90 degrees from, the y-axis). The visual axis A1 of a top-facing imaging element may be perpendicular to the visual axis A2 of the endoscope imaging element, as schematically depicted in FIG. 13A, and may also be perpendicular to the visual axis of any side imaging elements (indicated by dotted line A3). In some variations, a secondary imaging endoscopic device may not have any side imaging elements, and may only have a top imaging element, such as the secondary imaging endoscopic device 1300 depicted in FIGS. 13A-G. The secondary imaging endoscopic device 1300 may be used to examine the esophagus, the gastroesophageal junction, and/or other structures of the upper gastrointestinal tract. The secondary imaging endoscopic device 1300 may comprise an imaging module 1305 having a top-facing imaging element 1302 and a corresponding top-facing light source 1304. The light source 1304 may be distal or proximal to the imaging element 1302, as may be desirable. Although the secondary imaging endoscopic device 1300 does not have a fluid module or flush ports, other variations may have a fluid module or flush ports (e.g., the fluid modules described above). The secondary imaging endoscopic device 1300 may have a clip or C-shaped sleeve 1303 as described previously. When installed over the endoscope 1310 (FIGS. 13B-C), the distal-most edge of the clip 1301 may be proximal to the distal rim 1312 of the endoscope, as described previously. The height H2 of the secondary imaging endoscopic device 1300 may be from about 1.5 mm to about 4 mm, e.g., about 2 mm (FIG. 13E). The secondary imaging endoscopic device 1300 may also comprise a longitudinal tube or control cable 1306 that encloses an electrical conduit 1308 that includes the wiring and power for the imaging element 1302 and light source 1304. The electrical conduit 1308 may comprise a PCB substrate upon which the imaging element 1302 (e.g., CMOS or CCD sensor and/or any lens assembly, prism or filter set) and light source 1304 may be mounted. The optical configuration of the imaging element may be any of the optical configurations described and depicted in FIGS. 17A-17C. In some variations, the imaging element may comprise an image sensor and a prism. Optionally, an imaging element may also comprise a lens disposed between the image sensor and the prism. For example, FIG. 13F depicts one variation of an imaging element 1302a that comprises a prism 1320 disposed directly over an image sensor 1322. The prism 1320 may have the capability of magnifying and/or focusing images such that a separate lens assembly is not necessary. Optionally, the prism 1320 may comprise a filter (e.g., an infrared filter). FIG. 13G depicts another variation of an imaging element 1302b that comprises a prism 1330, an image sensor 1332 and a lens assembly 1334 disposed between the prism and the image sensor. Optionally, the prism 1330 may comprise a filter (e.g., an infrared filter). The height H2 of the secondary imaging endoscopic device may vary depending on whether the imaging element has a lens assembly. In some variations, an imaging element that comprises a prism without a separate lens assembly may have a smaller profile (e.g., lower height) than an imaging element that comprises a prism and a separate lens assembly. Any of the imaging elements described above may have an image sensor with only a prism or an image sensor with both a prism and lens assembly, as described above.

FIG. 16B is a side-view schematic depiction of the field of views of a top-facing imaging element 1610, and the field of view of the primary endoscope 1606 imaging element 1608. The viewing angle 1602 of the top-facing imaging element may be from about 120 degrees to about 160 degrees, e.g., about 130 degrees or 135 degrees. As depicted there, a top-facing imaging element may provide both top views and rear/retrograde views of the area around the endoscope. The viewing angle 1604 of the primary endoscope imaging element 1608 may be from about 125 degrees to about 155 degrees, e.g., about 140 degrees. The field of view of the top-facing imaging element and the endoscope main imaging element may overlap, where the angle over overlap may be from about 2 degrees to about 30 degrees, e.g., about 5 degrees, about 10 degrees. The top-facing imaging element 1610 may be located about 2 mm to about 5 mm from the distal-most end of the endoscope 501, e.g., about 5 mm. Images acquired by a top-facing imaging element that is relatively close to the distal end of the endoscope may be more intuitively interpreted by a practitioner who is simultaneously viewing videos taken from the main front-facing imaging element and the top-facing imaging element. Providing a degree of overlap between the images/videos acquired by the secondary top-facing and main front-facing imaging elements may also provide images that are readily understood by a practitioner.

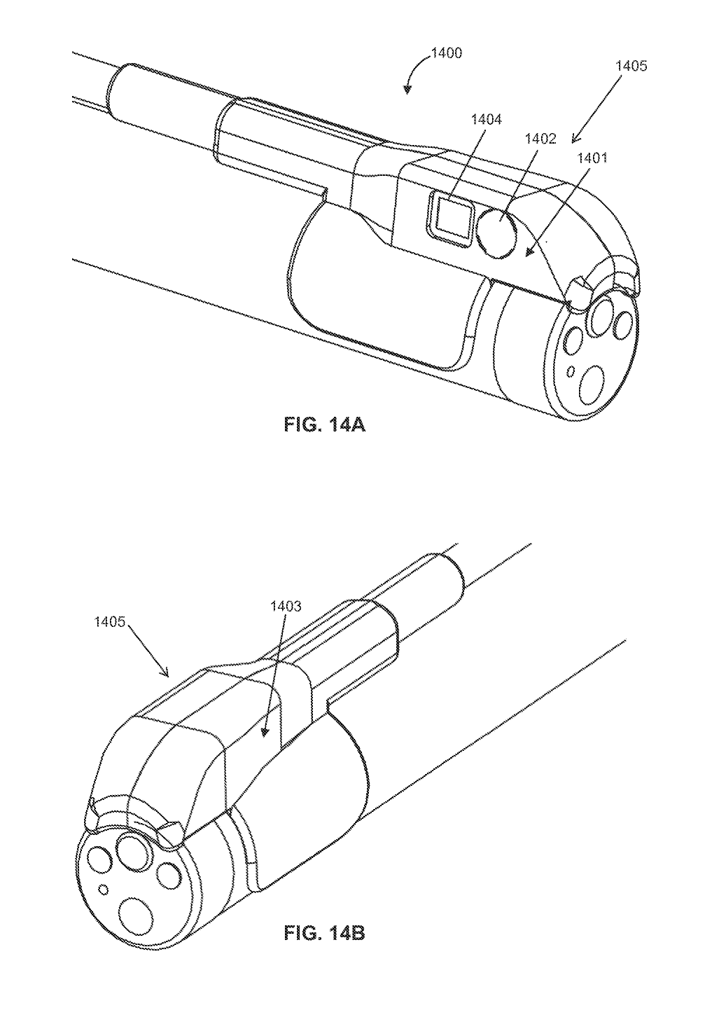

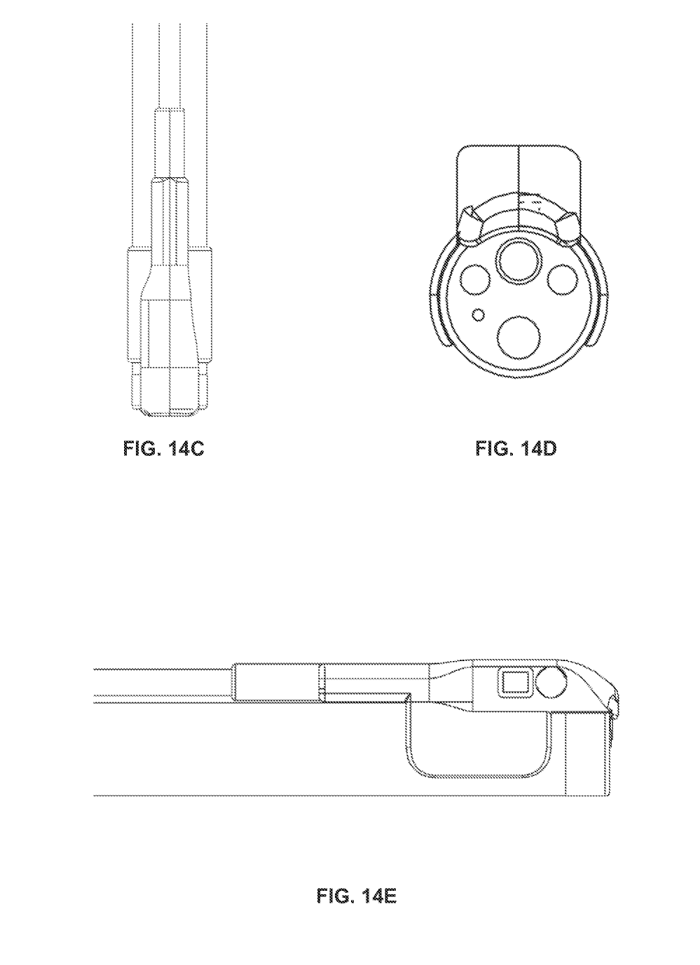

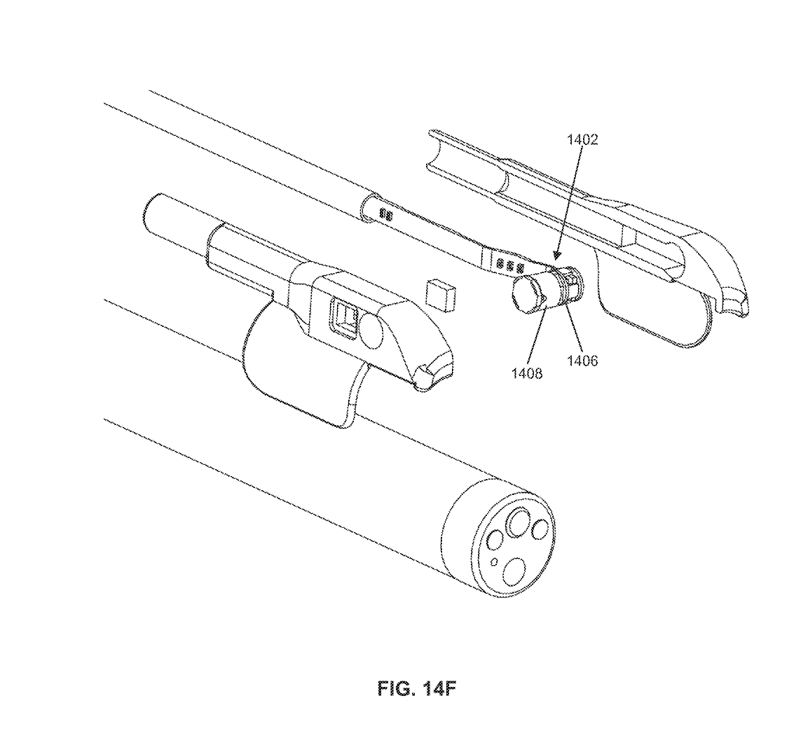

Other variations of a secondary imaging endoscopic device 1300 may comprise a single imaging element and corresponding light source, but the single imaging element and light source may be located on the side of the secondary imaging endoscopic device. One example of such a secondary imaging endoscopic device is depicted in FIGS. 14A-F. As depicted in FIG. 14A, secondary imaging endoscopic device 1400 may comprise an imaging module 1405 having a side-facing imaging element 1402 and a corresponding side-facing light source 104 on a first side 1401 of the secondary imaging endoscopic device 1400. A second side 1403 opposite the first side 1401 may not have any imaging element or light source (FIG. 14B). The secondary imaging endoscopic device 1400 may be used to examine the esophagus, the gastroesophageal junction, and/or other structures of the upper gastrointestinal tract, as will be further described below. FIG. 14F depicts an exploded view of the secondary imaging endoscopic device 1400. As depicted there, imaging element 1402 may comprise an image sensor 1406 and a lens assembly 1408 disposed in front of the image sensor. Alternatively or additionally, the imaging element 1402 may comprise a prism, as previously described. The optical configuration of the imaging element may be any of the optical configurations described and depicted in FIGS. 17A-17C. Other depicted features of the secondary imaging endoscopic device 1400 (e.g., the clip or C-shaped sleeve, any flush module or ports, control cable, electrical conduit, location of the imaging element with respect to the endoscope rim) may be similar to any of the embodiments described previously.

Any of the secondary imaging endoscopic devices described herein may be used with any desired endoscope to perform an examination of the lower gastrointestinal tract (e.g., in a colonoscopy procedure) or the upper gastrointestinal tract. One variation of a method for examining the upper gastrointestinal tract may comprise attaching a secondary imaging endoscopic device to the distal tip of an endoscope, advancing the endoscope under direct visualization over the tongue and through the esophagus, stomach and duodenum of a patient, and acquiring images of these structures, as well as images of the pylorus and the duodenum using the imaging elements of the secondary imaging endoscopic device and the endoscope. The images may include forward or antegrade views (e.g., using the imaging element of the endoscope), and/or lateral views (if the secondary imaging endoscopic device has one or more side-viewing imaging elements) and/or top views (if the secondary imaging endoscopic device has a top-viewing imaging element) and/or rear or retrograde view (e.g., as provided by any top-facing and/or side-facing imaging elements of a secondary imaging endoscopic device). The method may optionally comprise evaluating the pylorus, the duodenum (e.g., viewing the proximal duodenal bulb and the region around the sweep of the duodenum), GE junction, cardia or fundus to identify any evidence of neoplasm, ulceration or inflammation, polyps, and duodenal diverticula. The method may also comprise acquiring and/or viewing images of the major and minor duodenal papillae in the side/lateral view and/or top view to identify any regions of deformity or inflammation. Optionally, after viewing the anatomical regions of interest, the method may comprise obtaining biopsies by advancing a biopsy tool through a lumen of the endoscope. The endoscope may then be withdrawn through the gastroesophageal junction.

The secondary endoscopic imaging devices described herein may be used in diagnostic procedures (e.g., for imaging structures of the GI tract as part of a colonoscopy), such devices may also be used in therapeutic procedures. For example, a secondary endoscopic imaging device may be attached to a surgical device having a cutting, shearing, abrasion or lasso element for the removal of polyps identified in the colon or duodenum. In some variations, a secondary endoscopic imaging device may be attached to an endoscope with a working lumen for the insertion of surgical tools therethrough, so that a diagnostic procedure (e.g., imaging and identifying polyps) and a therapeutic procedure (e.g., removing any identified polyps) may be performed in the same session.

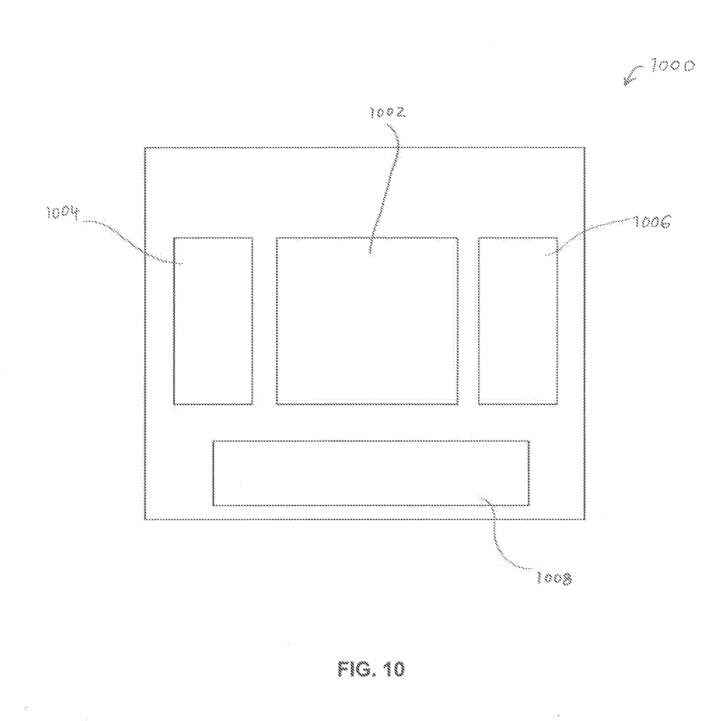

The visual output (e.g., still images and/or video) from any of the imaging elements of the main endoscope and/or the secondary imaging endoscopic device described herein may be displayed on one or more monitors in real-time. Optionally, the visual data from the imaging elements may be stored in computer memory for post-processing (e.g., for image stitching and/or reconstruction). In some variations, the image data from the main endoscope and the secondary imaging endoscopic device may be displayed on a single monitor or multiple monitors (e.g., one monitor for each imaging element). For example, as schematically depicted in FIG. 9A, a multi-imaging element endoscopy system 900 may comprise an endoscope having a main imaging element 902, a secondary imaging endoscopic device having a first side imaging element 904 and a second side imaging element 906, a controller having a first video processor 908 connected to the main imaging element 902, a second video processor 910 connected to the first side imaging element 904, a third video processor 912 connected to the second side imaging element 906, and a display 916. The first, second and third video processors may output video data using a digital visual interface (DVI) and may be directly connected to the display 916, or may be connected to a data relay 914, which is connected to the display 916. The controller may comprise these first, second and third video processors and the data relay, and may be enclosed within a single housing as a control unit. The controller may also comprise additional CPU and/or data processing and/or I/O devices (such as network devices), as may be desirable. The video processors may be synchronized such that the video images shown on the display correlate in time. For example, a sync signal 909 generated in the first video processor 908 may be transmitted to the second and third video processors 910, 912. In some variations, the sync signal 909 may be transmitted to the second video processor 910, which may then transmit a sync signal 909a to the third video processor 912 (e.g., connected in sequence or in series, such as is depicted in FIG. 9A) while in other variations, the sync signal 909 may be directly connected to both the second and third video processor 910, 912. The data relay 914 may optionally be connected to other devices that measure physiological parameters of the patient. For example, EKG data, blood pressure data, temperature data, breathing rate data, etc. may be measured by one or more devices 918 and transmitted to the data relay 914. Optionally, image data, such as MRI, CT, PET data, may be transmitted to the data relay and displayed on the display. The data relay 914 may parse that data and transmit it to the display 916. The display 916 may include a graphical user interface that allows a practitioner to control the type, format, layout, etc. of the information that is visualized on the display. The display may also be connected to a controller (as previously described), which may provide visual feedback regarding any user input (e.g., light levels of the light sources, activation of the imaging elements, display and/or operating modes, etc.). The images from all the imaging elements may be output onto the display and arranged in a way that reflect their relative locations and/or field of view to each other. In some variations, the image and/or video characteristics (e.g., color, contrast, hue, intensity, aspect ratio, scaling, resolution, etc.) from the different imaging elements may be adjusted so that they match and/or correspond to each other in an intuitive way. One example of the layout and types of information that may be output on a display is schematically represented in FIG. 10. The layout of display 1000 may comprise a central frame 1002 that may show the image/video from the main endo scope imaging element, a left frame 1004 that may show the image/video from the left side imaging element of the secondary imaging endoscopic device, and a right frame 1006 that may show the image/video from the right side imaging element of the secondary imaging endoscopic device. The arrangement of the images/video from each of the imaging elements may reflect the relative lateral position of the imaging elements on the endoscope and the secondary imaging endoscopic device. For example, the endoscope imaging element 902 may provide a central, forward view, the first side imaging element 904 may provide a left side/rear view, and the second side imaging element 906 may provide a right side/rear view. Therefore, the central frame 1002 may depict images/video from the endoscope imaging element, the right frame 1006 images/video from the right side imaging element, and the left frame 1004 images/video from the left side imaging element. In some variations, the aspect ratio of the images/video acquired by the side imaging elements may be adjusted to match the aspect ratio of the images/video acquired by the endoscope imaging element. For example, the images/video from the side imaging elements may be stretched in the vertical direction so that the images/video matches the vertical dimension of the images/video from the endoscope imaging element. Optionally, the display 1000 may also include a frame 1008 above or below the other frames that display other physiological data, including vital data (e.g., heart rate, blood pressure, etc.), EKG data, temperature data, breathing rate data, other image data (e.g., MRI, CT, PET, etc.) etc.

While the information layout depicted in FIG. 10 is illustrated for a single monitor, similar information and layout may be used in multiple monitors. For example, there may be one monitor corresponding to each imaging element, and the plurality of monitors may be arranged to reflect the relative positions and/or field of view to each other. Other physiological data (e.g., vital data, etc.) may be displayed on a central monitor or on all of the monitors. FIG. 9B depicts another variation of a multi-imaging element endoscopy system comprising an endoscope having a main imaging element 932, a secondary imaging endoscopic device having a first side imaging element 934 and a second side imaging element 936, a first video processor 938 connected to the main imaging element 932, a second video processor 940 connected to the first side imaging element 934, a third video processor 942 connected to the second side imaging element 936, a first display 944 connected to the first video processor 938, a second display 946 connected to the second video processor 940 and a third display 948 connected to the third video processor 942. The system controller may comprise these first, second and third video processors, along with other components, as described above. The video processors may be synchronized such that the video images shown on the displays correlate in time. For example, a sync signal 939 generated in the first video processor 938 may be transmitted to the second and third video processors 940, 942. In some variations, the sync signal 939 may be transmitted to the second video processor 940, which may then transmit a sync signal 939a to the third video processor 942 (e.g., connected in sequence or in series, such as is depicted in FIG. 9B) while in other variations, the sync signal 939 may be directly connected to both the second and third video processor 940, 942. The controller may optionally comprise a data relay to which the video processors may be connected (instead of the monitors), which may be connected to the first, second and third monitors. As with the system 900 depicted in FIG. 9B, there may be one or more devices 950 that measure physiological parameters of the patient, and such data may be displayed on the monitor and/or stored in memory. As described above, various physiological data, including vital data and image data from other imaging modalities (e.g., MRI, CT, PET, etc.) may be output to one or more of the displays. While the physiological data is depicted as being visualized on the first display, it should be understood that it may be visualized on any display and any number of displays. The displays may be arranged in a way that reflects the location and/or visual fields of the imaging elements relative to each other. For example, the endoscope imaging element 932 may provide a central, forward view, the first side imaging element 934 may provide a left side/rear view, and the second side imaging element 936 may provide a right side/rear view. Therefore, the first display 944 may be located in a central position, with the second display 946 to its right and the third display 948 to its left. In some variations, the displays may be vertically arranged to reflect the relative position of the imaging elements (e.g., the second and third displays 946, 948 may be arranged vertically higher than the first display 944). As described previously, the image/video characteristics of each of the imaging elements may be adjusted so that they correspond to each other, which may help a practitioner readily interpret the image/video data.

What is claimed as new and desired to be protected by Letters Patent of the United States is:

1. A detachable imaging device for use with an endo scope having a front-facing imaging element, the detachable imaging device comprising:
a clip configured to be releasably disposed over a distal portion of an endoscope, the clip comprising a proximal edge, a distal edge, an inner region and an outer region;
an imaging module attached to the outer region of the clip, the imaging module comprising a first side-facing imaging element having a first visual axis, a first side light source adjacent to the first imaging element such that the first light source provides illumination for the acquisition of images by the first imaging element, a second side-facing imaging element having a second visual axis that is co-linear with the visual axis of the first side imaging element, and a second side light source located adjacent to the second imaging element such that the second light source provides illumination for the acquisition of images by the second imaging element;
a control cable attached to the imaging module for powering and controlling the imaging module separately from the endoscope;
a fluid delivery module attached to the clip comprising a first outlet port, a second outlet port and an internal channel connecting the first and second outlet ports, wherein the first port is located proximal to the first side-facing imaging element and the second port is located proximal to the second side-facing imaging element, and wherein the first and second outlet ports are connected via an internal channel; and
a fluid conduit connected to the internal channel of the fluid delivery module, wherein the fluid conduit is located along an outer surface and along the length of the endoscope, and wherein the fluid conduit is configured to transport fluid to the fluid delivery module separately from the endoscope.

2. The detachable imaging device of claim 1, wherein the control cable is located along the outer surface and along the length of the endo scope.

3. The detachable imaging device of claim 1, wherein the first and second outlet ports are each located within first and second concave regions of the fluid delivery module.

4. The detachable imaging device of claim 3, wherein the concavity of the first and second concave regions are selected such that fluid from the first and second outlet ports are directed towards the first and second side-facing imaging elements.

5. The detachable imaging device of claim 1, wherein the fluid delivery module further comprises an inlet port in communication with the internal channel, wherein the fluid conduit is connected to the inlet port.

6. The detachable imaging device of claim 5, wherein the fluid conduit is detachable from the inlet port.

7. The detachable imaging device of claim 1, wherein the clip comprises an adhesive located along an endoscope-contacting surface.

8. The detachable imaging device of claim 1, wherein an endo scope-contacting surface of the clip comprises an elastomeric material.

9. The detachable imaging device of claim 1, wherein each of the first and second side-facing imaging elements of the imaging module comprises a lens assembly disposed over an image sensor.

10. The detachable imaging device of claim 9, wherein each of the first and second side-facing imaging elements comprises a prism in front of each of the lens assemblies.

11. The detachable imaging device of claim 1, wherein each of the first and second side-facing imaging elements of the imaging module comprises a prism disposed over an image sensor.

12. The detachable imaging device of claim 1, wherein the viewing angle for each of the first and second side-facing imaging elements is at least 135 degrees.

13. The detachable imaging device of claim 12, wherein the field of view of each of the first and second side-facing imaging elements overlaps with or is adjacent to the field of view of the front-facing imaging element.

14. The detachable imaging device of claim 1, further comprising a controller that is configured to combine images acquired by the first and second side-facing imaging element and the endoscope imaging element to simulate a continuous view.

15. The detachable imaging device of claim 14, wherein the controller is configured to stitch the images acquired by the first and second side-facing imaging element and the endo scope imaging element into a single image having a continuous view.

16. The detachable imaging device of claim 14, wherein the controller is configured to output the images acquired by the first and second side-facing imaging element and the endo scope imaging element to one or more display devices.

17. The detachable imaging device of claim 1, wherein the first side-facing imaging element and the second side-facing imaging element face in opposite directions.

18. The detachable imaging device of claim 1, wherein the fluid delivery module comprises a housing, wherein one or more portions of the housing is optically translucent.

19. The detachable imaging device of claim 1, wherein the housing of the fluid delivery module comprises one or more curves around the first and second outlet ports that forms a fluid dynamic path toward the first and second side-facing imaging elements for fluids exiting the first and second outlet ports.

20. The detachable imaging device of claim 1, wherein the first outlet port is on a first side of the fluid delivery module and the second outlet port is on a second side of the fluid delivery module opposite the first side, and wherein the internal channel spans across the fluid delivery module.

21. The detachable imaging device of claim 20, wherein the internal channel is a U-shaped cavity spanning between the first and second outlet ports.

22. The detachable imaging device of claim 1, wherein the first and second side-facing imaging elements are co-linear with a front-facing imaging element of the endo scope when the detachable imaging device is attached to the endoscope.

23. The detachable imaging device of claim 1, wherein the first and second side-facing imaging elements comprise an image sensor and a prism.

24. The detachable imaging device of claim 23, wherein the first and second side-facing imaging elements further comprise a lens assembly.

25. A detachable imaging device for use with an endo scope having a front-facing imaging element, the detachable imaging device comprising:
a clip configured to be releasably disposed over a distal portion of an endoscope;
an imaging module attached to the clip, the imaging module comprising a top-facing imaging element having a visual axis that is perpendicular to the circumference of the clip and the visual axis of a front-facing imaging element of the endoscope, and a top-facing light source located adjacent to the first imaging element such that the light source provides illumination for the acquisition of images by the imaging element; and
a control cable attached to the imaging module for powering and controlling the imaging module separate from the endoscope, wherein the control cable is located along an outer surface and along the length of the endo scope;
wherein the top-facing imaging element and the endoscope front-facing imaging element are configured to simultaneously acquire images with different fields of view, and wherein the field of view of the top-facing imaging element overlaps with the field of view of the endoscope front-facing imaging element.

26. The detachable imaging device of claim 25, wherein the clip comprises an adhesive located along an endoscope-contacting surface of the clip.

27. The detachable imaging device of claim 25, wherein an endoscope-contacting surface of the clip comprises an elastomeric material.

28. The detachable imaging device of claim 25, wherein top-facing imaging element comprises a lens assembly disposed over an image sensor.

29. The detachable imaging device of claim 28, wherein the top-facing imaging element comprises a prism in front of the lens assembly.

30. The detachable imaging device of claim 25, wherein the top-facing imaging element comprises a prism disposed over an image sensor.

31. The detachable imaging device of claim 25, wherein the viewing angle for the top-facing imaging element is at least 135 degrees.
